SEQUENCE LISTING (27 sequences)

SEQ ID NO: 1 - DNA, 12 bp, Artificial Sequence
  Description of Artificial Sequence Synthetic oligonucleotide
ggttagatctcc

SEQ ID NO: 2 - DNA, 12 bp, Artificial Sequence
  Description of Artificial Sequence Synthetic oligonucleotide
gttagatctcct

SEQ ID NO: 3 - DNA, 12 bp, Artificial Sequence
  Description of Artificial Sequence Synthetic oligonucleotide
ttagatctccta

SEQ ID NO: 4 - DNA, 12 bp, Artificial Sequence
  Description of Artificial Sequence Synthetic oligonucleotide
tagatctcctag

SEQ ID NO: 5 - DNA, 12 bp, Artificial Sequence
  Description of Artificial Sequence Synthetic oligonucleotide
aaatctcgtaga

SEQ ID NO: 6 - DNA, 12 bp, Artificial Sequence
  Description of Artificial Sequence Synthetic oligonucleotide
gatctcctagaa

SEQ ID NO: 7 - DNA, 12 bp, Artificial Sequence
  Description of Artificial Sequence Synthetic oligonucleotide
atatcctagaag

SEQ ID NO: 8 - DNA, 12 bp, Artificial Sequence
  Description of Artificial Sequence Synthetic oligonucleotide
tctcctagaaga

SEQ ID NO: 9 - DNA, 12 bp, Artificial Sequence
  Description of Artificial Sequence Synthetic oligonucleotide
ctcctagaagat

SEQ ID NO: 10 - DNA, 12 bp, Artificial Sequence
  Description of Artificial Sequence Synthetic oligonucleotide
tcgtagaagatc

SEQ ID NO: 11 - DNA, 12 bp, Artificial Sequence
  Description of Artificial Sequence Synthetic oligonucleotide
aaatctcctaga

SEQ ID NO: 12 - DNA, 12 bp, Artificial Sequence
  Description of Artificial Sequence Synthetic oligonucleotide
tcctagaagatc

SEQ ID NO: 13 - DNA, 95 bp, Homo sapiens
cacacacatcctaacactaccctaacacagccctaatctaaccctggccaacctgtctctcaacttaccctccattaccctgcctccactcgtta

SEQ ID NO: 14 - DNA, 92 bp, Homo sapiens
cacacacatcctaacactaccctaacacagccctaatttaaccctggccaacctgttcaacttaccctccattaccctgcctccactcgtta

SEQ ID NO: 15 - DNA, 92 bp, Homo sapiens
cacacacatcctaacactaccctaacacagccctaatctaaccctggccaacctgtctctcttaccctccattaccctgcctccattcgtta

SEQ ID NO: 16 - DNA, 69 bp, Homo sapiens
cacacacatcctaacactaccctaacacagccctaatctaaccctggccaacctgtctctcactcgtta

SEQ ID NO: 17 - DNA, 65 bp, Homo sapiens
cacacacatcctaacactaccctaacacagccctaatctaaccctggccaacctgtctctcatta

SEQ ID NO: 18 - DNA, 78 bp, Homo sapiens
cacacacatcctaacactaccctaacacagccctaatctaaccctggccaacctgtctctcaacttaccctccattta

SEQ ID NO: 19 - DNA, 82 bp, Homo sapiens
cacacacatcctaacactaccctaacacagccctaatctaaccctggccaacctgtctctcaacttaccctccattaccctg

SEQ ID NO: 20 - DNA, 84 bp, Homo sapiens
cacacacatcctaacactaccctaacacagccctaatctaaccctggccaacctgtctctcaacttaccctccattaccctgcc

SEQ ID NO: 21 - DNA, 87 bp, Homo sapiens
cacacacatcctaacactaccctaacacagccctaatctaaccctggccaacctgtctctcaacttaccctccattaccctgcctcc

SEQ ID NO: 22 - DNA, 94 bp, Homo sapiens
acacacatcctaacactaccctaacacagccctaatctaaccctggccaacctgtctctcaacttaccctccattaccctgcctccactcgtta

SEQ ID NO: 23 - DNA, 85 bp, Homo sapiens
ctaacactaccctaacacagccctaatctaaccctggccaacctgtctctcaacttaccctccattaccctgcctccactcgtta

SEQ ID NO: 24 - DNA, 76 bp, Homo sapiens
ccctaacacagccctaatctaaccctggccaacctgtctctcaacttaccctccattaccctgcctccactcgtta

SEQ ID NO: 25 - DNA, 68 bp, Homo sapiens
cagccctaatctaaccctggccaacctgtctctcaacttaccctccattaccctgcctccactcgtta

SEQ ID NO: 26 - DNA, 56 bp, Homo sapiens
aaccctggccaacctgtctctcaacttaccctccattaccctgcctccactcgtta

SEQ ID NO: 27 - DNA, 47 bp, Homo sapiens
caacctgtctctcaacttaccctccattaccctgcctccactcgtta

(19) United States
(12) Patent Application Publication
(10) Pub. No.: US 2020/0185055 A1
(43) Pub. Date: Jun. 11, 2020

(54) Methods and Systems for Nucleic Acid Variant Detection and Analysis

(71) Applicant: Cambridge Cancer Genomics Limited, Cambridge (GB)

(72) Inventors: Geoffroy DUBOURG-FELONNEAU, Alameda, CA (US); Luke HARRIES, Cambridge (GB); Harry CLIFFORD, Leeds (GB); Nirmesh PATEL, Sidcup (GB)

(21) Appl. No.: 16/752,240

(22) Filed: Jan. 24, 2020

Related U.S. Application Data
(63) Continuation of application No. PCT/US19/55885, filed on Oct. 11, 2019.
(60) Provisional application No. 62/745,196, filed on Oct. 12, 2018.

(51) Int. Cl. G16B 20/20; G16B 40/20; G16B 30/10; G06N 3/04; G06N 3/08

(52) U.S. Cl. CPC G16B 20/20; G16B 40/20; G06N 3/08; G06N 3/0454; G06N 3/0445; G16B 30/10

(57) ABSTRACT

Disclosed herein are methods, systems, and devices for detection of nucleotide variants. In some aspects, the methods, systems, and devices of the present disclosure can be used to detect germline variant or somatic variant in a biological sample, e.g., a sample from a tumor tissue. In other aspects, the methods, systems, and devices of the present disclosure can be used to detect somatic variant in cell-free nucleic acids from a biological sample, such as blood, plasma, serum, saliva, or urine. In some aspects, the methods, systems, and devices of the present disclosure make use of neural networks, such as convolutional neural networks for variant detection.

### CROSS-REFERENCE

This application is a continuation of International Patent Application No. PCT/US2019/055885, filed Oct. 11, 2019, which claims the benefit of U.S. Provisional Patent Application No. 62/745,196, filed Oct. 12, 2018, each of which is entirely incorporated herein by reference.

### BACKGROUND

Whole genome sequencing to identify genetic variants is becoming increasingly valuable in the clinical settings. Although the cost of such sequencing has decreased, it can still be too expensive for large-scale deployment, and understanding more complex genetic disorders can involve widespread population-scale sequencing and analysis. This can be achieved by accomplishing a greater accuracy of variant identification at lower depth sequencing. Thus, there remains an urgent need for improved methods and systems for variant detection. In some cases, performing variant calling on large input files corresponding to DNA sequence data can involve significant time and computational resources to complete. Further, in some cases, pileup images generated from DNA sequencing read data can be difficult to represent in such a manner as to be efficiently interpreted by machine learning algorithms (e.g., models) and/or humans.

### SUMMARY

In an aspect, the present disclosure provides a method for determining a somatic nucleotide variant in cell-free nucleic acids from a subject, comprising: (a) obtaining a plurality of sequencing reads of the cell-free nucleic acids from the subject and a plurality of sequencing reads of nucleic acids from a normal tissue of the subject; (b) generating a data input from the plurality of sequencing reads of the cell-free nucleic acids and the plurality of sequencing reads of the nucleic acids from the normal tissue; and (c) determining the somatic nucleotide variant in the cell-free nucleic acids by applying a trained neural network to the data input.

In some cases, the data input comprises one or more tensors, and each of the plurality of sequencing reads of the cell-free nucleic acids and each of the plurality of sequencing reads of the nucleic acids from the normal tissue are represented in a different row of the one or more tensors.

In another aspect, the present disclosure provides a method for determining a somatic nucleotide variant in cell-free nucleic acids from a subject, comprising: (a) obtaining a plurality of sequencing reads of the cell-free nucleic acids from the subject; (b) generating a data input comprising one or more tensors, wherein each of the plurality of sequencing reads of the cell-free nucleic acids is represented in a different row of the one or more tensors; and (c) determining the somatic nucleotide variant in the cell-free nucleic acids by applying a trained neural network to the data input.

In some cases, the method further comprises obtaining a plurality of sequencing reads from a normal tissue of the subject, wherein each of the plurality of sequencing reads of the cell-free nucleic acids is represented in a different row of the one or more tensors. In some cases, the one or more tensors comprise digital representations of images. In some cases, the images comprise RGB images.

In some cases, the cell-free nucleic acids are obtained or derived from a biological sample comprising one or more of: blood, serum, plasma, saliva, urine, and any combination thereof.

In another aspect, the present disclosure provides a method for determining a somatic nucleotide variant in nucleic acids from a tumor tissue of a subject, comprising: (a) obtaining a plurality of sequencing reads of the nucleic acids from the tumor tissue of the subject and a plurality of sequencing reads of nucleic acids from a normal tissue of the subject; (b) generating a data input comprising one or more tensors, wherein each of the plurality of sequencing reads of the nucleic acids from the tumor tissue and each of the plurality of sequencing reads of the nucleic acids from the normal tissue are represented in a different row of the one or more tensors, respectively; and (c) determining the somatic nucleotide variant in the nucleic acids from the tumor tissue by applying a trained neural network to the data input. In some cases, the one or more tensors comprise a first tensor and a second tensor.

In another aspect, the present disclosure provides a method for determining a somatic nucleotide variant in nucleic acids from a tumor tissue of a subject, comprising: (a) obtaining a plurality of sequencing reads of the nucleic acids from the tumor tissue of the subject and a plurality of sequencing reads of nucleic acids from a normal tissue of the subject; (b) generating a data input comprising a first tensor and a second tensor, wherein the first tensor comprises representation of the plurality of sequencing reads of the nucleic acids from the tumor tissue, and the second tensor comprises representation of the plurality of sequencing reads of nucleic acids from a normal tissue; and (c) determining the somatic nucleotide variant in the nucleic acids from the tumor tissue by applying a trained neural network to the first and second tensors. In some cases, each of the plurality of sequencing reads of the nucleic acids from the tumor tissue is represented in a different row of the first tensor, and each of the plurality of sequencing reads of the nucleic acids from the normal tissue is represented in a different row of the second tensor.

In another aspect, the present disclosure provides a method for determining a somatic nucleotide variant in nucleic acids from a tumor tissue of a subject, comprising: (a) obtaining a plurality of sequencing reads of the nucleic acids from the tumor tissue of the subject and a plurality of sequencing reads of nucleic acids from a normal tissue of the subject; (b) generating a data input from the plurality of sequencing reads of the nucleic acids from the tumor tissue and the plurality of sequencing reads of the nucleic acids from the normal tissue; and (c) determining the somatic nucleotide variant in the nucleic acids from the tumor tissue by applying a Siamese neural network to the data input, wherein the Siamese neural network comprises two trained sister neural networks, wherein each of the two identical trained sister neural networks generates an output, and wherein the Siamese neural network is configured to apply a function to outputs of the two identical trained sister neural networks to determine a classification indicative of whether the outputs are identical or different.

In another aspect, the present disclosure provides a method for determining a somatic nucleotide variant in nucleic acids from a tumor tissue of a subject, comprising: (a) obtaining a plurality of sequencing reads of the nucleic acids from the tumor tissue of the subject; (b) generating a data input from the plurality of sequencing reads of the nucleic acids from the tumor tissue and at least a portion of a reference genome of the subject; and (c) determining the somatic nucleotide variant in the nucleic acids from the tumor tissue by applying a trained neural network to the data input, wherein the trained neural network comprises a convolutional neural network configured to apply a kernel in a layer of the convolutional neural network to process at a fixed row of the kernel a representation of the at least a portion of the reference genome that is received from a preceding layer of the convolutional neural network.

In another aspect, the present disclosure provides a method for determining a somatic nucleotide variant in nucleic acids from a tumor tissue of a subject, comprising: (a) obtaining a plurality of sequencing reads of the nucleic acids from the tumor tissue of the subject; (b) generating a data input from the plurality of sequencing reads of the nucleic acids from the tumor tissue, wherein the data input is devoid of features extracted from the plurality of sequencing reads; and (c) determining the somatic nucleotide variant in the nucleic acids from the tumor tissue by applying a neural network directly to the data input.

In some cases, the method further comprises obtaining a plurality of sequencing reads of nucleic acids from a normal tissue of the subject. In some cases, the data input comprises representation of at least a portion of a reference genome of the subject. In some cases, the data input comprises one or more tensors, each of the plurality of sequencing reads of the nucleic acids from the tumor tissue and each of the plurality of sequencing reads of the nucleic acids from the normal tissue are represented in a different row of the one or more tensors, respectively. In some cases, the one or more tensors comprise digital representations of images. In some cases, the images comprise RGB images.

In some cases, the at least a portion of the reference genome is represented in at least one row of each of the one or more tensors. In some cases, the one or more tensors comprise a first tensor and a second tensor, wherein each of the plurality of sequencing reads of the cell-free nucleic acids is represented in a different row of the first tensor, and wherein each of the plurality of sequencing reads of the nucleic acids from the normal tissue is represented in a different row of the second tensor.

In some cases, the trained neural network comprises a Siamese neural network comprising two trained sister neural networks, wherein each of the two trained sister neural networks generates an output, and wherein the Siamese neural network is configured to apply a function to outputs from the two trained sister neural networks to determine a classification indicative of whether the outputs are identical or different. In some cases, the Siamese neural network is configured to determine the classification by comparing a distance between the outputs against a pre-determined threshold. In some cases, the threshold is pre-set in the Siamese neural network. In some cases, the threshold is optimized during training of the Siamese neural network. In some cases, the Siamese network comprises a fully connected layer applied to the distance.

In some cases, the trained neural network or the two trained sister neural networks comprise a deep neural network (DNN), a convolutional neural network (CNN), a feed forward network, a cascade neural network, a radial basis network, a deep feed forward network, a recurrent neural network (RNN), a long short-term memory (LSTM) network, a gated recurrent unit, an auto encoder, a variational auto encoder, a denoising auto encoder, a sparse auto encoder, a Markov chain, a Hopfiled network, a Boltzmann Machine, a restricted Boltzmann Machine, a deep belief network, a deconvolutional network, a deep convolutional inverse graphics network, a generative adversarial network, a liquid state machine, extreme learning machine, echo state network, deep residual network, a Kohonen network, a support vector machine, neural Turing machine, and any combination thereof. In some cases, the trained neural network or the two trained sister neural networks comprise a convolutional neural network (CNN), a recurrent neural network (RNN), a long short-term memory (LSTM) network, or any combination thereof. In some cases, the trained neural network or the two trained sister neural networks comprise a long short-term memory (LSTM) recurrent neural network (RNN). In some cases, the LSTM RNN comprises a bidirectional LSTM (BiLSTM) RNN.

In some cases, at least a portion of a reference genome of the subject is represented in the data input, wherein the trained neural network or the two trained sister neural networks comprise a convolutional neural network configured to apply a kernel in a layer of the convolutional neural network to process at a fixed row of the kernel a representation of the at least a portion of the reference genome that is received from a preceding layer of the convolutional neural network. In some cases, the method further comprises sequencing the cell-free nucleic acids, the nucleic acids from the normal tissue, or the nucleic acids from the tumor tissue.

In some cases, the plurality of sequencing reads of the nucleic acids from the tumor tissue, the plurality of sequencing reads of nucleic acids from the normal tissue, or the plurality of sequencing reads of the cell-free nucleic acids are within a genomic region covering a first genomic site. In some cases, the obtaining comprises determining the first genomic site as a potential site for nucleotide variant by applying a filter to the plurality of sequencing reads of the nucleic acids from the tumor tissue, the plurality of sequencing reads of nucleic acids from the normal tissue, or the plurality of sequencing reads of the cell-free nucleic acids. In some cases, applying the filter comprises comparing the plurality of sequencing reads of the nucleic acids from the tumor tissue, the plurality of sequencing reads of nucleic acids from the normal tissue, or the plurality of sequencing reads of the cell-free nucleic acids against a reference genome of the subject. In some cases, applying the filter further comprises calculating variant allele frequency for the first genomic site, and comparing the variant allele frequency against a pre-determined threshold.

In some cases, the trained neural network or the Siamese network is trained with a labeled dataset, wherein the labeled dataset comprises a plurality of sequencing reads labeled as having a somatic variant at a genomic site, and a plurality of sequencing reads labeled as having no somatic variant at the genomic site.

In some cases, the two trained sister neural networks are either (i) initialized with a germline variant training with a labeled dataset comprising a plurality of sequencing reads labeled as having a germline variant at a genomic site, and a plurality of sequencing reads labeled as having no somatic variant at the genomic site, or (ii) pre-set with weights initialized from the germline variant training. In some cases, the trained neural network, the Siamese network, or the two trained sister neural networks are trained with a labeled dataset comprising at least about 5,000, at least about 10,000, at least about 15,000, at least about 18,000, at least about 20,000, at least about 21,000, at least about 22,000, at least about 23,000, at least about 24,000, at least about 25,000, at least about 26,000, at least about 28,000, at least about 30,000, at least about 35,000, at least about 40,000, at least about 50,000, at least about 60,000, at least about 70,000, at least about 80,000, at least about 90,000, at least about 100,000, at least about 200,000, at least about 300,000, at least about 400,000, at least about 500,000, at least about 600,000, at least about 700,000, at least about 800,000, at least about 900,000, at least about 1,000,000, at least about 2,000,000, at least about 3,000,000, at least about 4,000,000, at least about 5,000,000, at least about 6,000,000, at least about 7,000,000, at least about 8,000,000, at least about 9,000,000, at least about 10,000,000, at least about 20,000,000, at least about 30,000,000, at least about 40,000,000, at least about 50,000,000, at least about 60,000,000, at least about 70,000,000, at least about 80,000,000, at least about 90,000,000, at least about 100,000,000, at least about 200,000,000, at least about 300,000,000, at least about 400,000,000, at least about 500,000,000, at least about 600,000,000, at least about 700,000,000, at least about 800,000,000, at least about 900,000,000, at least about 1,000,000,000, at least about 2,000,000,000, at least about 3,000,000,000, at least about 4,000,000,000, at least about 5,000,000,000, at least about 6,000,000,000, at least about 7,000,000,000, at least about 8,000,000,000, at least about 9,000,000,000, or at least about 10,000,000,000 labeled sequencing reads.

In some cases, the method further comprises generating a likelihood value corresponding to the determined somatic nucleotide variant in the nucleic acids from the tumor tissue. In some cases, generating the likelihood value comprises learning a probability density function over a set of weights of the trained neural network or the two trained sister neural networks. In some cases, learning the probability density function comprises applying Bayesian inference to the set of weights. In some cases, the trained neural network or the two trained sister neural networks comprise a BiLSTM RNN comprising two layers of BiLSTM cells. In some cases, the two layers of BiLSTM cells comprise variational dense layers or standard layers. In some cases, the likelihood value is a probability value of the determined somatic nucleotide variant being present in the tumor tissue of the subject.

In another aspect, the present disclosure provides a method, comprising detecting a somatic variant in a subject according to the method provided herein, and diagnosing, prognosticating, or monitoring a cancer in the subject. In some cases, the method further comprises providing treatment recommendations for the cancer. In some cases, the cancer is selected from the group consisting of: adrenal cancer, anal cancer, basal cell carcinoma, bile duct cancer, bladder cancer, cancer of the blood, bone cancer, a brain tumor, breast cancer, bronchus cancer, cancer of the cardiovascular system, cervical cancer, colon cancer, colorectal cancer, cancer of the digestive system, cancer of the endocrine system, endometrial cancer, esophageal cancer, eye cancer, gallbladder cancer, a gastrointestinal tumor, hepatocellular carcinoma, kidney cancer, hematopoietic malignancy, laryngeal cancer, leukemia, liver cancer, lung cancer, lymphoma, melanoma, mesothelioma, cancer of the muscular system, Myelodysplastic Syndrome (MDS), myeloma, nasal cavity cancer, nasopharyngeal cancer, cancer of the nervous system, cancer of the lymphatic system, oral cancer, oropharyngeal cancer, osteosarcoma, ovarian cancer, pancreatic cancer, penile cancer, pituitary tumors, prostate cancer, rectal cancer, renal pelvis cancer, cancer of the reproductive system, cancer of the respiratory system, sarcoma, salivary gland cancer, skeletal system cancer, skin cancer, small intestine cancer, stomach cancer, testicular cancer, throat cancer, thymus cancer, thyroid cancer, a tumor, cancer of the urinary system, uterine cancer, vaginal cancer, vulvar cancer, and any combination thereof. In some embodiments, (b) comprises processing a sequence alignment map (SAM) or binary alignment map (BAM) file of the plurality of sequencing reads of the cell-free nucleic acids or a SAM or BAM file of the plurality of sequencing reads of the nucleic acids from the normal tissue by: splitting the SAM or BAM file into a plurality of distinct microSAM or microBAM files, wherein each distinct microSAM or microBAM file comprises a different set from among a plurality of sets of the sequencing reads, and performing parallel processing of the plurality of distinct microSAM or microBAM files. In some embodiments, the method further comprises creating the plurality of sets of the plurality of sequencing reads, such that each set of the plurality of sets comprises all genomic regions represented in the plurality of sequencing reads that are closer to each other than a pre-determined maximum interval. In some embodiments, (b) comprises creating a pileup image from the plurality of sequencing reads of the cell-free nucleic acids; creating a normal pileup image from the plurality of sequencing reads of the nucleic acids from the normal tissue; applying a coloration algorithm to the pileup image and the normal pileup image; and concatenating the pileup image and the normal pileup image, thereby generating a pileup matrix.

In another aspect, the present disclosure provides a device for determining a somatic nucleotide variant in nucleic acids from a tumor tissue of a subject, comprising means for performing a method as disclosed herein.

In another aspect, the present disclosure provides a non-transitory computer readable medium storing computer program instructions for determining a somatic nucleotide variant in nucleic acids from a tumor tissue of a subject, the computer program comprising instructions which, when executed by a processor, cause the processor to perform operations according to a method as disclosed herein.

In another aspect, the present disclosure provides a computer-implemented method for performing parallel processing of a plurality of sequencing reads, comprising: (a) obtaining a sequence alignment map (SAM) file of the plurality of sequencing reads; (b) creating a plurality of sets of the plurality of sequencing reads, wherein each set of the plurality of sets comprises all genomic regions represented in the plurality of sequencing reads that are closer to each other than a pre-determined maximum interval; (c) splitting the SAM or BAM file into a plurality of distinct microSAM or microBAM files, wherein each distinct microSAM or microBAM file comprises a different set from among the plurality of sets of the sequencing reads; and (d) performing parallel processing of the plurality of distinct microSAM or microBAM files to identify genetic variants in the plurality of sequencing reads. In some cases, the method further comprises determining a coverage of the SAM or BAM file. In some cases, (d) comprises one or more of: overwriting read groups, indexing the SAM or BAM file or the plurality of distinct microSAM or microBAM files, and generating pileups from the plurality of distinct microSAM or microBAM files. In some cases, (d) comprises identifying somatic genetic variants in the plurality of sequencing reads, germline genetic variants in the plurality of sequencing reads, false-positive genetic variants arising from sequencing error, or a combination thereof.

In another aspect, the present disclosure provides a system for performing parallel processing of a plurality of sequencing reads, comprising: a database that is configured to store a sequence alignment map (SAM) or binary alignment map (BAM) file of the plurality of sequencing reads; and one or more computer processors operatively coupled to the database, wherein the one or more computer processors are individually or collectively programmed to: (i) create a plurality of sets of the plurality of sequencing reads, wherein each set of the plurality of sets comprises all genomic regions represented in the plurality of sequencing reads that are closer to each other than a pre-determined maximum interval; (ii) split the SAM or BAM file into a plurality of distinct microSAM or microBAM files, wherein each distinct microSAM or microBAM file comprises a different set from among the plurality of sets of the sequencing reads; and (iii) perform parallel processing of the plurality of distinct microSAM or microBAM files to identify genetic variants in the plurality of sequencing reads.

In another aspect, the present disclosure provides a non-transitory computer readable medium comprising machine-executable code that, upon execution by one or more computer processors, implements a method for performing parallel processing of a plurality of sequencing reads, the method comprising: (a) obtaining a sequence alignment map (SAM) or binary alignment map (BAM) file of the plurality of sequencing reads; (b) creating a plurality of sets of the plurality of sequencing reads, wherein each set of the plurality of sets comprises all genomic regions represented in the plurality of sequencing reads that are closer to each other than a pre-determined maximum interval; (c) splitting the SAM or BAM file into a plurality of distinct microSAM or microBAM files, wherein each distinct microSAM or microBAM file comprises a different set from among the plurality of sets of the sequencing reads; and (d) performing parallel processing of the plurality of distinct microSAM or microBAM files to identify genetic variants in the plurality of sequencing reads.

In another aspect, the present disclosure provides a computer-implemented method for generating a pileup matrix, comprising: (a) obtaining a plurality of sequencing reads of cell-free nucleic acids from a subject and a plurality of sequencing reads of nucleic acids from a normal tissue of the subject; (b) creating a pileup image from the plurality of sequencing reads of the cell-free nucleic acids; (c) creating a normal pileup image from the plurality of sequencing reads of the nucleic acids from the normal tissue; (d) applying a coloration algorithm to the pileup image and the normal pileup image; and (e) concatenating the pileup image and the normal pileup image, thereby generating the pileup matrix.

In some cases, creating the pileup image or the normal pileup image comprises applying a Concise Idiosyncratic Gapped Alignment Report (CIGAR) correction. In some cases, the method further comprises applying a quality filter to the pileup image and the normal pileup image. In some cases, the method further comprises adding a reference to the pileup image and the normal pileup image. In some cases, the method further comprises processing the pileup matrix to identify genetic variants in the plurality of sequencing reads of the cell-free nucleic acids or the plurality of sequencing reads of the nucleic acids from the normal tissue.

In another aspect, the present disclosure provides a system for generating a pileup matrix, comprising: a database that is configured to store a plurality of sequencing reads of cell-free nucleic acids from a subject and a plurality of sequencing reads of nucleic acids from a normal tissue of the subject; and one or more computer processors operatively coupled to the database, wherein the one or more computer processors are individually or collectively programmed to: (i) create a pileup image from the plurality of sequencing reads of the cell-free nucleic acids; (ii) create a normal pileup image from the plurality of sequencing reads of the nucleic acids from the normal tissue; (iii) apply a coloration algorithm to the pileup image and the normal pileup image; and (iv) concatenate the pileup image and the normal pileup image, thereby generating the pileup matrix. In some cases, creating the pileup image or the normal pileup image comprises applying a Concise Idiosyncratic Gapped Alignment Report (CIGAR) correction. In some cases, the method further comprises applying a quality filter to the pileup image and the normal pileup image. In some cases, the method further comprises adding a reference to the pileup image and the normal pileup image. In some cases, the method further comprises processing the pileup matrix to identify genetic variants in the plurality of sequencing reads of the cell-free nucleic acids or the plurality of sequencing reads of the nucleic acids from the normal tissue.

In another aspect, the present disclosure provides a non-transitory computer readable medium comprising machine-executable code that, upon execution by one or more computer processors, implements a method for generating a pileup matrix, the method comprising: (a) obtaining a plurality of sequencing reads of cell-free nucleic acids from a subject and a plurality of sequencing reads of nucleic acids from a normal tissue of the subject; (b) creating a pileup image from the plurality of sequencing reads of the cell-free nucleic acids; (c) creating a normal pileup image from the plurality of sequencing reads of the nucleic acids from the normal tissue; (d) applying a coloration algorithm to the pileup image and the normal pileup image; and (e) concatenating the pileup image and the normal pileup image, thereby generating the pileup matrix.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows an example of a flow chart for a method of variant detection.

FIG. 2 shows an example workflow of an implementation of a variant caller, GermlineNET.

FIG. 3 shows an example of computer code implementing an example of a method of encoding a base sequence to RGB.

FIG. 4 shows an example of computer code implementing an example of a method of encoding quality score to RGB.

FIG. 5 shows examples of pileup images of sequenced normal DNA with a reference generated by GermlineNET.

FIG. 6 is a diagram depicting an example of a basic structure of a Siamese network.

FIG. 7 shows an example of a workflow of an implementation of a somatic variant caller, SomaticNET.

FIG. 8 shows examples of pileup images of sequenced normal DNA and tumor DNA with a reference generated by SomaticNET.

FIG. 9 shows an example of a normal image and an example of a spliced image at the base layer of GermlineNET

FIG. 10 shows a computer system that can be programmed or otherwise configured to implement methods of the present disclosure.

FIG. 11 shows a diagram of an example of the methods and systems of the present disclosure.

FIG. 12 is a plot of a validation loss of ResNet34 vs. a learning rate (e.g., a number of epochs on a log10 scale), which was used for finding the initial learning rate during an example of training of GermlineNET.

FIG. 13 summarizes an example of training of ResNet34 with different dropouts, which produces varying validation loss vs. a number of epochs.

FIG. 14 is a plot of a validation loss of ResNet34 vs. a number of epochs for the first fold of cross-validation of GermlineNET.

FIG. 15 shows examples of pairs of images used for the omniglot verification task.

FIG. 16 shows a Siamese distance distribution from tumor and normal tissue classes in a validation set, showing that the model was able to learn to distinguish between the two classes.

FIG. 17 is a plot of validation accuracy vs. different Siamese distances.

FIG. 18A shows a visual representation of the pair matrix with w=10 and d=10.

FIG. 18B shows an example of results produced by the Bayesian long short-term memory (LSTM)-based approach applied to original data (left) and masked data (right).

FIG. 18C shows histograms of the output probabilities from the Bayesian neural network (BNN) tested on in-distribution samples (left) and masked data that are out-of-distribution (right).

FIG. 19 shows an example workflow of the machine learning-based variant calling, including obtaining tumor and normal sequencing files as inputs, encoding the tumor reads and normal reads into a pileup image, and performing variant calling by applying deep learning classification to the pileup image to generate a variant call format (VCF) file.

FIG. 20A shows an example of how simulated variants were spiked in silico into the NA12878 cell line dataset, which included sequencing cell line data to produce a plurality of sequencing reads, extracting and editing a known subset of the plurality of sequencing reads, and realigning the edited sequencing reads and returning them to the plurality of sequencing reads.

FIG. 20B shows an example of how high-confidence variants were identified from the MC3 (multi-center mutation calling in multiple cancers) dataset across technical replicates, which included re-sequencing multi-center tumor samples, performing variant calling in multiple sets of samples, and confirming the high-confidence variants.

FIG. 21A-21B show results demonstrating that the neural network achieved a very high AUROC on both datasets.

FIG. 22 shows that SomaticNET demonstrated superior performance in catching “edge cases” (e.g., borderline cases such as somatic variants that can be difficult to distinguish from germline variants or false-positive variants arising from sequencing error) as compared to Bayesian variant calling alone.

FIG. 23A shows an example of splitting an original SAM or BAM file into N different microSAM or microBAM files.

FIG. 23B shows an example of how a list of region sets is generated as part of the parallelized processing of DNA sequence data.

FIGS. 24A-24E show examples of pileup images that can be challenging for humans to visually interpret.

### DETAILED DESCRIPTION

Overview

In an aspect, the present disclosure relates to methods, systems, and devices for detecting nucleotide variants, e.g., germline variants or somatic variants, in a biological sample. In some cases, the methods, systems, and devices of the present disclosure can also be used for diagnosing, prognosticating, and monitoring a disease or condition of a subject, e.g., cancer, by the detection of the nucleotide variants.

Determining nucleic acid variants, e.g., somatic variants, e.g., DNA mutations that have occurred in a tumor, can have dramatic impact in many clinical settings. For example, it can vastly change cancer diagnosis, treatment, and management. For example, the mutation profile of a subject can enable targeted chemotherapy and detection of resistance to treatment months earlier than other methods.

DNA can be the physical storage for a sequence of four bases {A,T,C,G} which can control cell life, death, and replication. DNA mutations can result in substitutions, insertions, or deletions of this sequence. The progressive accumulation of these mutations in particular regions of DNA can then result in various pathological conditions, for example, uncontrolled cell replication that can eventually lead to development of cancer.

To determine which mutations have occurred in a pathological tissue of a subject, one approach can be to extract nucleic acids from the pathological tissue and compare it with nucleic acids from a normal tissue (e.g., healthy tissue) of the same subject. The comparison can be performed using sequencing reads of the nucleic acids, for instance, sequencing reads obtained from next-generation sequencing (NGS) of the nucleic acids. However, there can be challenges to this approach. Some sequencing technologies, such as some NGS platforms, do not directly sequence the nucleic acids themselves. Instead, in some cases, the nucleic acids can be replicated (e.g., in some of the cases of RNA, the reverse transcripts of the RNAs can be replicated) and then broken into a large number of fragments which can then be sequenced. Thereafter, a sequencing read can be generated for each sequenced fragment. In some cases, to reconstruct the original DNA sequence, the sequencing reads are reassembled by aligning them to a reference nucleic acid sequence, e.g., a DNA sequence. Therefore, a high error rate can therefore be introduced by some sequencing technologies, such as NGS.

The task of detecting whether there is a DNA mutation at a particular site as described herein can be seen as a classification task and termed as “variant calling”. While many tools may use different heuristics with vastly different results, the methods, systems, and devices of the present disclosure can make use of neural networks for variant calling, which can include comparing the sequencing reads against a reference sequence, e.g., a reference genome of the subject and distinguishing a nucleotide variant present in the real DNA sequence from an artificially introduced error in the sequencing reads.

In some cases, a method for determining a nucleotide variant in nucleic acids from a tissue of a subject can comprise obtaining a plurality of sequencing reads of the nucleic acids from the tissue of the subject; generating a data input from the plurality of sequencing reads of the nucleic acids; and determining the nucleotide variant in the nucleic acids from the tissue using a trained neural network applied to the data input. While some somatic variant callers rely upon heuristics and statistics which assume that read errors are independent, the neural networks as described herein can be configured to learn the likelihood function of read errors and to apply the likelihood function to distinguish true nucleotide variants from artifacts (e.g., artificial read errors).

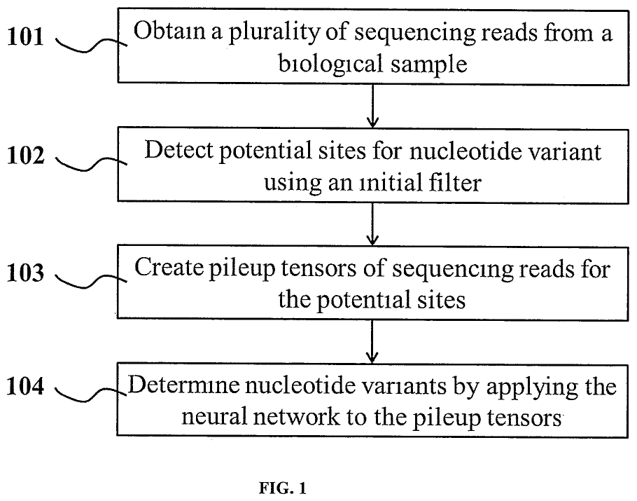

One example of the methods provided herein is illustrated in the flow chart in FIG. 1. As shown in the figure, the method can comprise obtaining a plurality of sequencing reads from a biological sample (as in operation 101); detecting potential sites for nucleotide variant using an initial filter (as in operation 102); creating pileup tensors of sequencing reads for the potential sites (as in operation 103); and determining the nucleotide variants by applying a neural network to the pileup tensors (as in operation 104).

In some cases, the methods, systems, and devices of the present disclosure can also detect somatic variants versus germline variants by making use of neural networks. Germline variants, as described herein, can refer to nucleotide variants inherited from the parents of a subject. In some cases, each germline variant can be present throughout the cells in the body. The germline variant can be a single base substitution, which can be termed as a Single Nucleotide Polymorphism (SNP). On average, SNPs can occur at approximately 1 in every 300 bases and as such there can be about 10 million SNPs in the human genome. Somatic variants as described herein can refer to variants that occur spontaneously in the cells (e.g., somatic cells) of a body, and in some cases somatic variants can be only present in the affected cell and their progeny.

In some instances, a method for determining a somatic variant in nucleic acids from a subject can comprise obtaining a plurality of sequencing reads of the nucleic acids from the tissue of the subject; generating a data input from the plurality of sequencing reads of the nucleic acids; and determining the somatic nucleotide variant in the nucleic acids from the tissue using a trained neural network applied to the data input. In some cases, the neural networks as described herein can be configured to distinguish true nucleotide variants from artifacts (e.g., artificial read errors) and determine whether a detected nucleotide variant is a germline variant or not. In some cases, the detected nucleotide variant that is not a germline variant can be called as a somatic variant by the neural network.

Variant Caller

In another aspect, the present disclosure provides methods, systems, and devices for variant calling, e.g., detection of nucleotide variants in nucleic acids from a biological sample.

In some cases, the method comprises obtaining a plurality of sequencing reads of the nucleic acids from a biological sample. In some cases, the method further comprises generating a data input from the plurality of sequencing reads. The data input as provided herein can comprise one or more tensors that comprise a representation of the plurality of sequencing reads. For instance, each of the sequencing reads can be stored directly in the form of A, T, G, or C, in a different row of the tensor. In some cases, the sequencing reads are encoded in a certain manner. In some examples, the one or more tensors comprise other information of the sequencing reads, for instance, a quality score for each nucleotide of the reads, or other features that can be extracted from the reads. In some examples, the one or more tensors are devoid of features extracted from the sequencing reads.

In some cases, the sequencing reads from a sequencer are filtered before conversion into the data input for the variant calling. For example, the sequencing reads can be assembled against a reference genome and grouped according to their genomic locations. In some cases, the reads are compared against a reference genome, and potential sites for variants and reads containing the potential variants are detected. There can be many different filters for this selection process. Many of the applicable filters can have varying complexity and be derived from simple heuristics or statistical models, and an arbitrary threshold can be set for such filtering processes. However, in some cases, the sequencing reads are not filtered before conversion into the data input for the variant calling, and no potential variant sites are selected for the subsequent analysis.

In some cases, the data input as described herein, e.g., the one or more tensors, are pileup tensors of the plurality of sequencing reads. For example, each of the sequencing reads can be represented in a different row of the tensor. One non-limiting and intuitive example of such a tensor is a pileup digital image. The pileup digital image can comprise pixels, each of which represents a nucleotide of a read, and each row of the image represents a different read. As such, a bioinformatic classification task can be converted into an image classification task. While some variant callers consider the candidate site in isolation from its neighboring bases, the methods of the present disclosure can use the pileup tensors, e.g., pileup images, to include and analyze nearby errors or variants. In some cases, the tensor can further comprise a representation of at least a portion of the reference genome. In some non-limiting examples, in the input tensor, the sequencing reads, and optionally the reference genome, are aligned according to their genomic location. Thus, each nucleotide of a sequencing read can have a corresponding reference nucleotide on the same column of the tensor, regardless whether they are the same or different (e.g., due to a sequencing error or genetic variance).

However, in some cases, the data input may not comprise a representation of a reference genome, for instance, in one or more separate rows from the sequencing reads. For instance, in some cases, the representation of sequencing reads comprises information on the difference between the sequencing reads and the reference genome on each genomic location. In some cases, the data input also comprises a representation of sequencing reads from different sources. For example, sequencing reads from the parent(s) of the subject can be encoded in the data input, or sequencing reads from a biological sample of the subject can be obtained a different time point.

A neural network can also be termed as artificial neural network (ANN). Neural networks that can be used in the methods, systems, and devices of the present disclosure can include a deep neural network (DNN), a convolutional neural network (CNN), a feed forward network, a cascade neural network, a radial basis network, a deep feed forward network, a recurrent neural network (RNN), a long short-term memory (LSTM) network (e.g., a bi-directional LSTM (BiLSTM) network), a gated recurrent unit, an auto encoder, a variational auto encoder, a denoising auto encoder, a sparse auto encoder, a Markov chain, a Hopfiled network, a Boltzmann Machine, a restricted Boltzmann Machine, a deep belief network, a deconvolutional network, a deep convolutional inverse graphics network, a generative adversarial network, a liquid state machine, extreme learning machine, echo state network, deep residual network, a Kohonen network, a support vector machine, neural Turing machine, and any combination thereof.

Convolutional Neural Networks (CNNs) can be a specialized type of neural network which can be well suited for image classification tasks. CNNs can be transitionally invariant, e.g., they can learn to extract the feature regardless of location. A network can be regarded as a CNN if, in at least one layer, a convolution instead of matrix multiplication is used. The convolution for X, an m×n image with c channels can be a differential function defined as follows

$${S ( X )}_{[ i , j , k ]} = X * {K}_{[ i , j , k ]} = {\sum }_{m} {\sum }_{n} {\sum }_{c} {X}_{[ m , n , c ]} {K}_{[ i - m ] , j - {n}_{v} c - k ]} \qquad ( 1 )$$

where K is the kernel, K is a p×q matrix with c channels.

The convolution layer in the network can take a similar form to the multi-layer perceptron

f⁽ⁱ⁾(X)=σ(S(X)=b) (2)

where σ is a non-linearity and b is the bias.

In some cases, p and q, which denote the height and width of K, can be much smaller than m and n of X. For example, a 3×3×c shape for K can be used. The same kernel can be applied to multiple areas of the image which, when compared to a fully connected layer, can result in many fewer parameters, e.g., as in parameter sharing. Therefore, there can be fewer parameters to be optimized, thereby making the image easier to train, and the features can be detected in different locations, thereby achieving translational invariance.

The kernel can be regarded as a filter which can extract a particular feature. In some cases, the kernel weights can be chosen manually, such as by using an edge detector. In other cases, with back-propagation, the kernel weights can be learned. In some cases, successive convolutions can thus select for high-level representations.

An example of an implementation of the methods described herein is GermlineNET, a deep learning-based variant caller. GermlineNET can be useful for detection of germline variants in a biological sample, but it can also be used to detect any type of genetic variant (e.g., germline variant, somatic variant, or both) in a biological sample. Germline variant calling can determine whether the differences, at a particular position i on the reference genome, between the reference base and the bases observed in sequencing are due to a true biological variant (e.g., heterozygous alternate or homozygous alternate) or an error (e.g., amplification, sequencing, alignment, or base calling error).

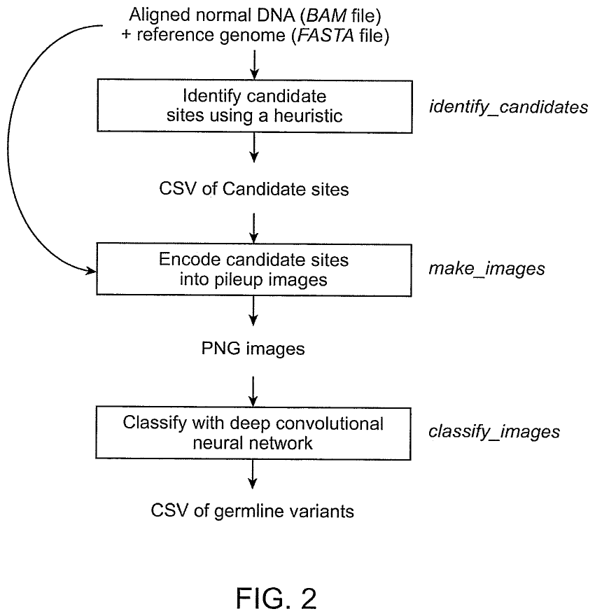

GermlineNET can be a data pipeline comprising three modules running sequentially, as shown in FIG. 2. When trained, GermlineNET can receive two inputs: the aligned germline DNA as a binary alignment map (BAM) file, obtained from sequencing the DNA of normal patient cells, and the reference genome to which it was aligned. GermlineNET can output a list of germline variants. The modules may include one or more of:
1. identify_candidates: The candidate sites can be identified using a frequency heuristic.
2. make_images: An RGB-encoded pileup image can be created for each candidate site.
3. classify_images: A deep convolutional network classifies the difference between the candidate site and the reference in the image as being (0) an error due to sequencing or (1) caused by a germline variant.

In order to reduce the number of sites that the deep learning model needs to classify, an initial filter with high sensitivity and low specificity can be used. In this case, a simple heuristic—Variant Allele Frequency (VAF) threshold of 5% is used. VAF can be equal to the percentage of bases at that location which do not match the reference and can be calculated using:

$$VAF = \frac{mismatches}{total reads} \qquad ( 3 )$$

If there was no error introduced in sequencing, one can expect the homozygous reference VAF≈0%, the heterozygous variant VAF 50% and the homozygous variant VAF≈100%. A VAF threshold of 5% can have high sensitivity and low specificity, e.g., it can capture the majority of variant sites and still dramatically reduce the number of sites to classify further. This heuristic can be implemented in the function make csv( . . . ) which can take two inputs: the path to the sequenced DNA file (e.g., BAM file, SAM file, or CRAM file), and the path to the reference DNA file (FASTA file), to generate a CSV file.

GermlineNET can encode the pileup tensor into an RGB image. The image can be considered a tensor X ∈ = [symbol] ^{m×n×c}. Each row X_{i,l,l} of the tensor can represent a different sequencing read. In this case, the first five rows of the tensor are filled with the replicated reference genome. Each column X_{l,j,l} of the tensor can represent a position on the reference genome. Each layer X_{l,l,k} of the tensor can represent a channel. There can be two channels. The first channel X_{l,}_{^{∞}}_{,0} can contain an encoding of the base at that position. The second channel X_{l,l,1} can contain the quality score of the base in that position. An example of channels one and two before encoding are shown below:

$$\begin{matrix}X ∈ {X}_{t} {X}_{1 {,}^{*} , 0} [ \begin{matrix}A T T C G \\ A T T\end{matrix} ] , {X}_{bb 1} = [ \begin{matrix}60 \; 60 \; 60 \; 60 \; 60 \\ 54 \; 65 \; 54\end{matrix} ] & ( 4 ) \\ ⌊ \begin{matrix}C T C \\ T C G\end{matrix} ] ⌊ \begin{matrix}34 \; 43 \; 50 \\ 47 \; 46 \; 39\end{matrix} ⌋ & \end{matrix}$$

As seen in Equation 4, there can be many overlapping sequencing reads. This can provide multiple observations for each base which serve to corroborate or disprove the presence of a genetic variant, and additionally, variants and errors at nearby sites can provide further evidence as to whether there is a mutation or error at i. This information can be learned as a feature by the CNN.

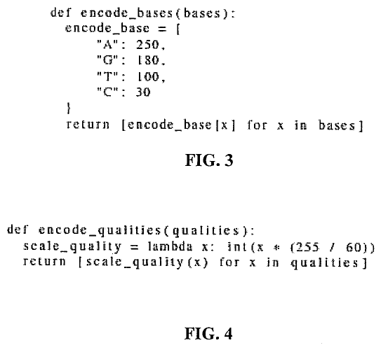

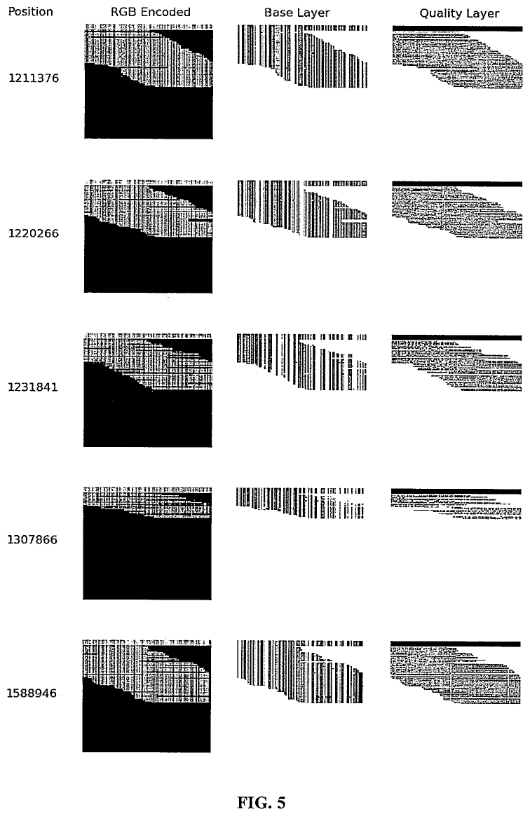

EncodedPileup can be a class for creating the encoded pileup as an image. Initiating the class can use the path to the sequenced DNA, the path to the reference DNA, or the location of the site in question (the chromosome and position). For each row, the base and the quality score can be encoded to RGB values—integers between 0-256. The base sequence can be encoded using the function shown in FIG. 3. Additionally, the quality scores for the read, normally a sequence of values between 30 and 60, can be scaled using the following function shown in FIG. 4. Examples of the generated images are shown in FIG. 5.

In GermlineNET, two classes over which a probability distribution can be generated are shown in Table 1.

TABLE 1
Deep CNN classes to predict

| # | Class | Variants |
|---|---|---|
| 0 | Not germline variant | Homozygous normal |
| 1 | Germline variant | Heterozygous/homozygous alternate |

Next, Inception, ResNet, and weight initialization can be used to build the neural network that processes the data input and gives out classification outputs (0 or 1 as shown in Table 1). For the final layer of the neural network, the softmax function can be used, and for the loss function, the negative log-likelihood can be used. The combination of softmax and negative log-likelihood can be used for classification tasks where the number of classes k ∈ [symbol] , k>0.

The softmax function can be defined as follows:

$${softmax ( z )}_{i} = \frac{exp ( {z}_{i} )}{{\sum }_{j} exp ( {z}_{j} )} \qquad ( 5 )$$

The softmax function can be a generalization of the sigmoid function, and can transform the CNN output vector z ∈ [symbol] ^{k} to y ∈ [0,1]^{k}, where Σᵢyᵢ=1. Therefore, y can be considered a probability distribution.

The negative log likelihood can be defined as:

NLL(y)ᵢ=−log(y)i (6)

The NLL can penalize the network for low confidence in the correct class. The negative sign can make the log-likelihood convex and, therefore, it can be minimized (locally) using gradient descent.

Somatic Variant Caller

In another aspect, the present disclosure provides methods, systems, and devices for somatic variant calling, e.g., detection of somatic nucleotide variants in nucleic acids from a biological sample.

In some cases, the method described herein comprises obtaining a plurality of sequencing reads of the nucleic acids from the biological sample and a plurality of sequencing reads of nucleic acids from a different normal tissue of the subject. In some cases, the method comprises generating a data input from both the sequencing reads of the nucleic acids from the biological sample and the plurality of sequencing reads of nucleic acids from the normal tissue. The somatic nucleotide variant can then be determined using a trained neural network applied to the data input. Normal tissue as described herein can refer to any tissue of the subject that is considered to be healthy or not suspected to carry the same somatic variants as in the biological sample. For instance, when a tumor tissue in liver is analyzed for potential somatic variants (mutations), as a reference, nucleic acids from normal tissues can be, for example, buccal DNA or DNA from forearm skin.

In some cases, the data input comprises one or more tensors. For example, the data input can comprise 1, 2, 3, 4, 5, 6, 7, 8, 9, 10, or more than 10 tensors. Each of the plurality of sequencing reads of the nucleic acids from the tumor tissue and each of the plurality of sequencing reads of the nucleic acids from the tumor tissue can be represented in a different row of the one or more tensors, respectively. In some cases, the data input comprises a first tensor and a second tensor. The first and second tensors can each comprise a representation of the sequencing reads from the biological sample and the normal tissue, respectively. Alternatively, the data input can comprise only one tensor, and such a single tensor can comprise a representation of sequencing reads of nucleic acids from both the biological sample and the normal tissue.

In some cases, somatic nucleotide variants can be determined by first detecting variants from each set of sequencing reads, e.g., reads of nucleic acids from the biological sample and sequencing reads of nucleic acids from the normal tissue. Variants detected from sequencing reads of nucleic acids from the normal tissue can be considered as germline variants, which can then compared against the variants detected from sequencing reads of nucleic acids from the biological sample. In other words, somatic variant calling can be reframed as a verification task. Verification tasks can involve determining a binary classification: whether two unseen images are (0) of the same type or (1) of a different type. For example, in facial verification, two unseen photos of faces can be classified as (0) of the same person, or (1) of a different person. In some cases, in somatic variant calling, at a particular site, the underlying DNA of the normal DNA and the sequenced DNA can be (0) the same or (1) different, due to a somatic mutation.

In some cases, a method for determining a somatic nucleotide variant in nucleic acids from a tumor tissue of a subject comprises use of a Siamese neural network applied to the data input. A Siamese neural network can comprise two identical trained sister neural networks, each of which can generate an output, and the Siamese neural network can be configured to apply a function to the outputs from the two trained sister neural networks to classify whether the two outputs are the same or different.

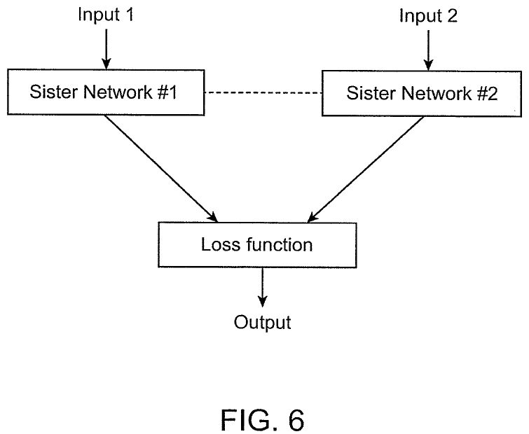

A Siamese network can be a high-level neural network architecture used for classification tasks with few training samples of each class. A Siamese network can transform two input vectors into vectors in a latent space with inputs (0) of the same type close together, and inputs (2) of different types further apart. Referring to FIG. 6, an example of a Siamese network architecture is shown. The two sister networks can have the same weights and structures. Therefore, passing each input vector through the sister network can be considered as a forward pass of each vector through the same network. The loss function can penalize the network for inputs (0) of the same type being far apart, and for inputs (1) of different types being close together.

In some cases, the distance function used is the Euclidean Distance

EucledianDistance=l₂=|f(x₀)−f(x₁|)² (7)

with f being the sister network. The loss can be calculated using the Contrastive Loss.

ContrasstiveLoss=(1−y)½l₂)²+y½ max(0,m−(l₂)² (8)

When x⁽⁰⁾≈x⁽¹⁾, y=0. If the network classifies as far apart, l₂ can be large, resulting in a large loss. However, if the network classifies them close together, l₂ can be small, resulting in a small loss.

When x⁽⁰⁾≠x⁽¹⁾, y=1. If the network classifies as far apart, l₂ can be large, therefore, max 0,m−(l₂)² can be small or zero, resulting in a small loss. However, if the network classifies them close together, l₂ can be small, resulting in a large m−(l₂)² resulting in a small loss.

As Loss(f(x)) can be differentiable, the gradient of the error with respect to the parameters can be calculated with back propagation, and used to update the weights of the network with SGD.

In some cases, the Siamese neural network is configured to classify whether the two outputs are the same or different by comparing the distance between the two outputs against a threshold. The threshold can be preset in the Siamese neural network, e.g., set at a certain number based on heuristics. Alternatively, the threshold can be optimized during training of the Siamese neural network. In some other cases, the Siamese network can further comprise a fully connected layer applied to the distance.

An example of an implementation of somatic variant calling according to some aspects of the present disclosure is SomaticNET, a deep learning-based somatic variant caller. SomaticNET was developed by using a Siamese network adapted from GermlineNET's CNN.

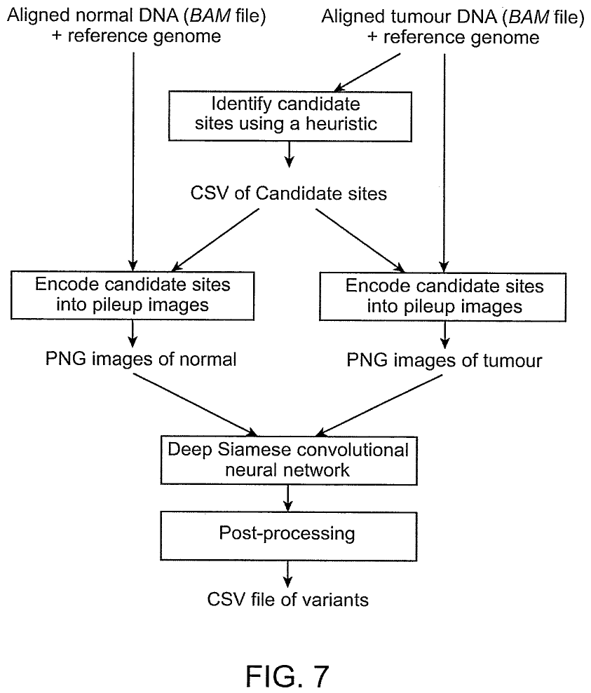

SomaticNET can be a data pipeline similar to GermlineNET, comprising three modules, as shown in FIG. 7. When trained, SomaticNET can accept three inputs: the aligned germline DNA as a BAM file, obtained from sequencing the DNA of normal patient cells, the aligned tumor DNA as a BAM file, and the reference genome to which they were both aligned to. SomaticNET can then output a CSV file of somatic variants. The three modules can include:
1. identify_candidates: The candidate sites can be identified using a heuristic. The same heuristic can be used as GermlineNET; however, it can be applied to the sequenced tumor BAM file.
2. make_images: An RGB-encoded pileup image can be created for each candidate site. This can be the same module as GermlineNET.
3. classify_images: A Siamese CNN can classify the differences between the sequenced normal DNA and the sequenced tumor DNA as being (0) an error due to sequencing or (1) caused by a somatic variant.

To reduce the number of sites that the deep learning model needs to classify, a heuristic can be used as an initial filter. As with GermlineNET, the VAF threshold can be used in this case; however, in SomaticNET the heuristic can be applied to the tumor DNA. This can result in candidate SNPs being selected along with candidate somatic variant sites. However, the trained neural network can learn only to call the somatic variants.

In this case, the VAF threshold value can be calibrated for a high sensitivity and low specificity. If the value used for the VAF threshold is too low, too many false sites can be passed through and be selected as candidates. If the value is too high, too many SNV sites can be missed. In this case, a VAF threshold of 0.02 was selected, as it can capture the majority of the union but exclude a large percentage of the uncalled sites.

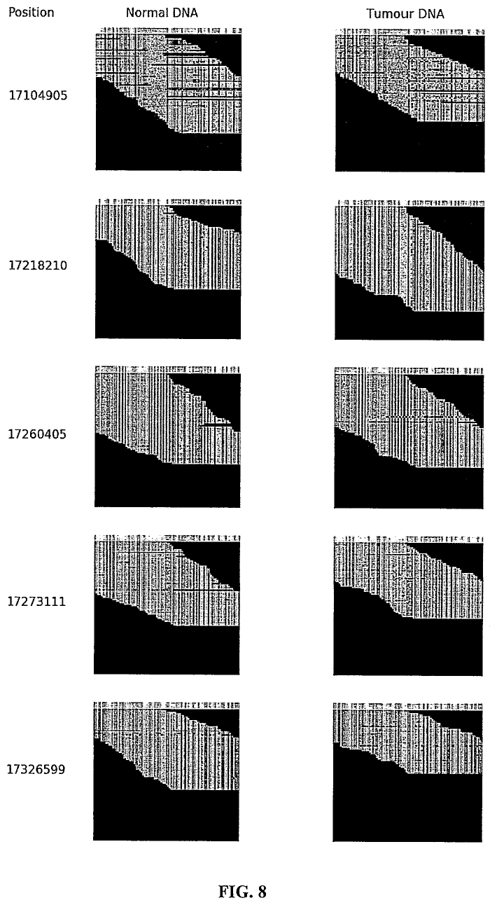

To generate the pileups, GermlineNET's make_images can be reused here to create the pileup matrix and encode it into an RGB image. For each candidate site, one image can be created using the normal DNA and the reference, and one image using the tumor DNA and the reference, as shown in FIG. 5. FIG. 8 shows examples of pileup images generated in SomaticNET for sequenced normal and tumor DNAs.

SomaticNET's neural network can be a Siamese convolutional neural network. In SomaticNET's Siamese CNN, the sister network can be GermlineNET's CNN, with the weight initialized from SNP training.

The two sister networks each can output a vector of size 4×1. The Euclidean distance between the two vectors can be calculated and trained with the Contrastive Loss Function, to learn that the two vectors should have no distance between them when there is (0) no mutation present in the tumor sample's candidate site, and that there should be a large distance between them when there is (1) a mutation present in the tumor sample's candidate site.

Additionally, a fully connected layer and the sigmoid function can be built in to follow the distance between the two output vectors. This can result in a scalar output. An output of 0 can be defined to mean they were of the same type—the differences at the candidate sites can be errors. An output of 1 can be defined to mean they were different—the differences at the candidate sites can be due to a somatic variant.

Spliced Kernel

In some cases, a spliced kernel approach is used in the methods of the present disclosure. In some cases, reference genome or a portion thereof is represented in the data input. As in some cases, the representation of the reference genome is only present in some rows of the tensor, e.g., a few top rows of the tensor. In some cases, the CNN can only extract features which the kernel is applied to, and the reference genome, in some cases, can only be compared with the normal DNA at the border.

The Spliced Kernel approach can keep the reference at a constant position on the kernel. This can allow the Kernel to learn the weights needed to appropriately combine the two pieces of information (e.g., the reference DNA and the normal DNA). In some cases, when applying the convolution, the reference can be dynamically inserted at the site on the kernel. Alternatively, the data input, e.g., the image can be altered such as to insert the reference at every s rows, where s is the vertical stride of the convolutional kernel, with the restriction that s>1. FIG. 9 illustrates a spliced image, which is an example of an implementation of the second approach. In the GermlineNET and SomaticNET as discussed above, with the spliced images generated first, optimized PyTorch convolutional layers can then be used. To implement the spliced image, the python package NumPy can be used to apply a series of transformations.

Training of the Neural Network

In another aspect, the present disclosure provides methods of training the neural network as described herein. Model training for supervised learning can involve data with high confidence labels. In another aspect, the present disclosure provides a labeled dataset for training of neural networks to detect nucleotide variants, e.g., germline variants or somatic variants.

In some cases, the neural network as provided herein is trained with a labeled dataset which comprises a number of sequencing reads, each of which can be labeled as having a nucleotide variant (1) or not having a nucleotide variant (0) at a predetermined genomic site. In some cases, the labeled dataset can have sequencing reads that are labeled for different genomic sites. In some cases, the labeled dataset can have one group of sequencing reads that are labeled for one genomic site. In some cases, the label can be indicative of whether the sequencing read has a nucleotide variant (1) or does not have a nucleotide variant (0). In some cases, the label can have more information besides the presence or absence of a nucleotide variant, for instance, the type of the variance, e.g., substitution, deletion, or insertion, or the mutant or variant nucleotide (e.g., A, T, G, or C).

In some cases, the neural network provided herein is trained with a labeled dataset comprising sequencing reads labeled with information of germline variants. In some cases, the neural network provided herein is trained with a labeled dataset comprising sequencing reads labeled with information of somatic variants. In some cases, the neural network provided herein is trained with a labeled dataset comprising sequencing reads labeled with information of both germline variants and somatic variants.

In some examples, the neural network provided herein is trained with a labeled dataset comprising at least about 5,000, at least about 10,000, at least about 15,000, at least about 18,000, at least about 20,000, at least about 21,000, at least about 22,000, at least about 23,000, at least about 24,000, at least about 25,000, at least about 26,000, at least about 28,000, at least about 30,000, at least about 35,000, at least about 40,000, at least about 50,000, at least about 60,000, at least about 70,000, at least about 80,000, at least about 90,000, at least about 100,000, at least about 200,000, at least about 300,000, at least about 400,000, at least about 500,000, at least about 600,000, at least about 700,000, at least about 800,000, at least about 900,000, at least about 1,000,000, at least about 2,000,000, at least about 3,000,000, at least about 4,000,000, at least about 5,000,000, at least about 6,000,000, at least about 7,000,000, at least about 8,000,000, at least about 9,000,000, at least about 10,000,000, at least about 20,000,000, at least about 30,000,000, at least about 40,000,000, at least about 50,000,000, at least about 60,000,000, at least about 70,000,000, at least about 80,000,000, at least about 90,000,000, at least about 100,000,000, at least about 200,000,000, at least about 300,000,000, at least about 400,000,000, at least about 500,000,000, at least about 600,000,000, at least about 700,000,000, at least about 800,000,000, at least about 900,000,000, at least about 1,000,000,000, at least about 2,000,000,000, at least about 3,000,000,000, at least about 4,000,000,000, at least about 5,000,000,000, at least about 6,000,000,000, at least about 7,000,000,000, at least about 8,000,000,000, at least about 9,000,000,000, or at least about 10,000,000,000 sequencing reads labeled as having the nucleotide variant (e.g., germline variant or somatic variant) at a given genomic site, and at least about 5,000, at least about 10,000, at least about 15,000, at least about 18,000, at least about 20,000, at least about 21,000, at least about 22,000, at least about 23,000, at least about 24,000, at least about 25,000, at least about 26,000, at least about 28,000, at least about 30,000, at least about 35,000, at least about 40,000, at least about 50,000, at least about 60,000, at least about 70,000, at least about 80,000, at least about 90,000, at least about 100,000, at least about 200,000, at least about 300,000, at least about 400,000, at least about 500,000, at least about 600,000, at least about 700,000, at least about 800,000, at least about 900,000, at least about 1,000,000, at least about 2,000,000, at least about 3,000,000, at least about 4,000,000, at least about 5,000,000, at least about 6,000,000, at least about 7,000,000, at least about 8,000,000, at least about 9,000,000, at least about 10,000,000, at least about 20,000,000, at least about 30,000,000, at least about 40,000,000, at least about 50,000,000, at least about 60,000,000, at least about 70,000,000, at least about 80,000,000, at least about 90,000,000, at least about 100,000,000, at least about 200,000,000, at least about 300,000,000, at least about 400,000,000, at least about 500,000,000, at least about 600,000,000, at least about 700,000,000, at least about 800,000,000, at least about 900,000,000, at least about 1,000,000,000, at least about 2,000,000,000, at least about 3,000,000,000, at least about 4,000,000,000, at least about 5,000,000,000, at least about 6,000,000,000, at least about 7,000,000,000, at least about 8,000,000,000, at least about 9,000,000,000, or at least about 10,000,000,000 sequencing reads labeled as not having the nucleotide variant (e.g., germline variant or somatic variant) at the given genomic site.

In some examples, the neural network provided herein is trained with a labeled dataset comprising about 5,000, about 10,000, about 15,000, about 18,000, about 20,000, about 21,000, about 22,000, about 23,000, about 24,000, about 25,000, about 26,000, about 28,000, about 30,000, about 35,000, about 40,000, about 50,000, about 60,000, about 70,000, about 80,000, about 90,000, about 100,000, about 200,000, or about 1,000,000 sequencing reads labeled as having the nucleotide variant (e.g., germline variant or somatic variant) at a given genomic site, and about 5,000, about 10,000, about 15,000, about 18,000, about 20,000, about 21,000, about 22,000, about 23,000, about 24,000, about 25,000, about 26,000, about 28,000, about 30,000, about 35,000, about 40,000, about 50,000, about 60,000, about 70,000, about 80,000, about 90,000, about 100,000, about 200,000, or about 1,000,000 sequencing reads labeled as not having the nucleotide variant (e.g., germline variant or somatic variant) at the given genomic site.

In some examples, the neural network provided herein is trained with a labeled dataset comprising about 5,000 to about 10,000, about 10,000 to about 15,000, about 15,000 to about 18,000, about 18,000 to about 20,000, about 20,000 to about 21,000, about 21,000 to about 22,000, about 22,000 to about 23,000, about 23,000 to about 24,000, about 24,000 to about 25,000, about 25,000 to about 26,000, about 26,000 to about 28,000, about 28,000 to about 30,000, about 30,000 to about 35,000, about 35,000 to about 40,000, about 40,000 to about 50,000, about 50,000 to about 60,000, about 60,000 to about 70,000, about 70,000 to about 80,000, about 80,000 to about 90,000, about 90,000 to about 100,000, about 100,000 to about 200,000, about 200,000 to about 1,000,000, or more than about 1,000,000 sequencing reads labeled as having the nucleotide variant (e.g., germline variant or somatic variant) at a given genomic site. In some examples, the neural network provided herein is trained with a labeled dataset comprising about 5,000 to about 10,000, about 10,000 to about 15,000, about 15,000 to about 18,000, about 18,000 to about 20,000, about 20,000 to about 21,000, about 21,000 to about 22,000, about 22,000 to about 23,000, about 23,000 to about 24,000, about 24,000 to about 25,000, about 25,000 to about 26,000, about 26,000 to about 28,000, about 28,000 to about 30,000, about 30,000 to about 35,000, about 35,000 to about 40,000, about 40,000 to about 50,000, about 50,000 to about 60,000, about 60,000 to about 70,000, about 70,000 to about 80,000, about 80,000 to about 90,000, about 90,000 to about 100,000, about 100,000 to about 200,000, about 200,000 to about 1,000,000, or more than about 1,000,000 sequencing reads labeled as not having the nucleotide variant (e.g., germline variant or somatic variant) at a given genomic site.

In some examples, the neural networks provided herein can be trained with a publicly available dataset, such as Genome In A Bottle (GIAB). In some aspects, the present disclosure provides dataset of labeled sequencing reads. For example, a dataset of labeled sequencing reads is provided for training neural networks to detect somatic variants.

In some cases, a semi-simulated dataset can be generated and used for model training and evaluation. This type of dataset can be generated in-silico by altering the bases at a particular position of a BAM file until the desired VAF is achieved. In some cases, however, this dataset is not realistic for at least two possible reasons: (a) the generated mutations may falsely assume independence between read errors; (b) the generated mutations may only affect the base at the mutation site, not the quality score. Some variant callers assume that the read errors are independent, with the true likelihood function being unknown. In some examples of the present disclosure, however, deep learning, e.g., Siamese networks are used to learn the likelihood function of read error occurrence and mutation occurrence, by reframing the problem of variant calling as a verification task. As a result, training data that follows the true likelihood function can be needed.

In some cases, the neural network may be trained such that a desired accuracy of variant calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The accuracy of variant calling may be calculated as the percentage of potential variants that are correctly identified or classified as being or not being a variant of interest (e.g., somatic variant or germline variant).

In some cases, the neural network may be trained such that a desired sensitivity of variant calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The sensitivity of variant calling may be calculated as the percentage of true biological variants (e.g., not false-positive variants arising from sequencing error) that are correctly identified or classified as being a variant of interest (e.g., somatic variant or germline variant).

In some cases, the neural network may be trained such that a desired specificity of variant calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The specificity of variant calling may be calculated as the percentage of non-variants that are correctly identified or classified as not being a variant of interest (e.g., somatic variant or germline variant).

In some cases, the neural network may be trained such that a desired positive predictive value (PPV) of variant calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The PPV of variant calling may be calculated as the percentage of identified or called variants that correspond to a true biological variant.

In some cases, the neural network may be trained such that a desired negative predictive value (NPV) of variant calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The NPV of variant calling may be calculated as the percentage of identified or called non-variants that truly correspond to a non-biological variant.

Sequencing

In some aspects, the methods and systems of the present disclosure can further comprise sequencing nucleic acids from a biological sample.

Any available sequencing technique can be used to generate the sequencing reads used in the methods and systems of the present disclosure. For example, sequencing can include next-generation sequencing (NGS). In some instances, sequencing nucleic acids can be performed using chain termination sequencing, hybridization sequencing, Illumina sequencing (e.g., using reversible terminator dyes), ion torrent semiconductor sequencing, mass spectrophotometry sequencing, massively parallel signature sequencing (MPSS), Maxam-Gilbert sequencing, nanopore sequencing, polony sequencing, pyrosequencing, shotgun sequencing, single molecule real time (SMRT) sequencing, SOLiD sequencing (hybridization using four fluorescently labeled di-base probes), universal sequencing, or any combination thereof.

A sequencing read obtained using methods and systems of the present disclosure can refer to a string of nucleotides sequenced from any part or all of a nucleic acid molecule. For example, a sequencing read can be a short string of nucleotides (e.g., 20-150) complementary to a nucleic acid fragment, a string of nucleotides complementary to an end of a nucleic acid fragment, or a string of nucleotides complementary to an entire nucleic acid fragment that exists in the biological sample. Nucleotides can include adenine (A), thymine (T), cytosine (C), and guanine (G) in DNA; or adenine (A), uracil (U), cytosine (C), and guanine (G) in RNA.

A sequencing read as described herein can comprise any appropriate length (e.g., number of consecutive nucleotides). In some cases, the sequencing reads comprise at least 20, at least 30, at least 40, at least 50, at least 60, at least 70, at least 80, at least 90, at least 100, at least 110, at least 120, at least 130, at least 135, at least 140, at least 145, at least 150, at least 155, at least 160, at least 165, at least 170, at least 175, at least 180, at least 190, at least 200, at least 220, at least 240, at least 260, at least 280, at least 300, at least 320, at least 340, at least 360, at least 380, at least 400, at least 500, at least 600, at least 700, at least 800, at least 900, at least 1,000, at least 2,000, at least 3,000, at least 4,000, at least 5,000, at least 6,000, at least 7,000, at least 8,000, at least 9,000, at least 10,000, at least 15,000, at least 20,000, at least 25,000, at least 30,000, at least 35,000, at least 40,000, at least 45,000, at least 50,000, at least, 55,000, or at least 60,000 consecutive nucleotides. In some cases, the sequencing reads comprise about 20, about 30, about 40, about 50, about 60, about 70, about 80, about 90, about 100, about 110, about 120, about 130, about 135, about 140, about 145, about 150, about 155, about 160, about 165, about 170, about 175, about 180, about 190, about 200, about 220, about 240, about 260, about 280, about 300, about 320, about 340, about 360, about 380, about 400, about 500, about 600, about 700, about 800, about 900, about 1,000, about 2,000, about 3,000, about 4,000, about 5,000, about 6,000, about 7,000, about 8,000, about 9,000, about 10,000, about 15,000, about 20,000, about 25,000, about 30,000, about 35,000, about 40,000, about 45,000, about 50,000, about, 55,000, or about 60,000 consecutive nucleotides. In some cases, the sequencing reads comprise at most 20, at most 30, at most 40, at most 50, at most 60, at most 70, at most 80, at most 90, at most 100, at most 110, at most 120, at most 130, at most 135, at most 140, at most 145, at most 150, at most 155, at most 160, at most 165, at most 170, at most 175, at most 180, at most 190, at most 200, at most 220, at most 240, at most 260, at most 280, at most 300, at most 320, at most 340, at most 360, at most 380, at most 400, at most 500, at most 600, at most 700, at most 800, at most 900, at most 1,000, at most 2,000, at most 3,000, at most 4,000, at most 5,000, at most 6,000, at most 7,000, at most 8,000, at most 9,000, at most 10,000, at most 15,000, at most 20,000, at most 25,000, at most 30,000, at most 35,000, at most 40,000, at most 45,000, at most 50,000, at most 55,000, or at most 60,000 consecutive nucleotides.

The sequencing reads obtained using methods and systems of the present disclosure can be obtained at various sequencing depth. In some cases, the methods and systems of the present disclosure improve the sensitivity of variant calling and thus reduce the depth needed for accurate variant calling. Sequencing depth can refer to the number of times a locus is covered by a sequencing read aligned to the locus. The locus can be as small as a nucleotide, or as large as a chromosome arm, or as large as the entire genome. Sequencing depth can be about 5×, 10×, 15×, 20×, 25×, 30×, 35×, 40×, 45×, 50×, 60×, 70×, 80×, 90×, 100×, 150×, 200×, etc., where the number before “×” refers to the number of times a locus is covered with a sequencing read. Sequencing depth can be at least 5×, 10×, 15×, 20×, 25×, 30×, 35×, 40×, 45×, 50×, 60×, 70×, 80×, 90×, 100×, 150×, 200×, etc. Sequencing depth can be at most 5×, 10×, 15×, 20×, 25×, 30×, 35×, 40×, 45×, 50×, 60×, 70×, 80×, 90×, 100×, 150×, 200×, etc. Sequencing depth can also be applied to a plurality of loci, or the whole genome, in which case x can refer to the mean number of times the plurality of loci or the haploid genome, or the whole genome, respectively, is sequenced.

In some cases, the sequencing depth may be adjusted such that a desired accuracy of variant calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The accuracy of variant calling may be calculated as the percentage of potential variants that are correctly identified or classified as being or not being a variant of interest (e.g., somatic variant or germline variant).

In some cases, the sequencing depth may be adjusted such that a desired sensitivity of variant calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The sensitivity of variant calling may be calculated as the percentage of true biological variants (e.g., not false-positive variants arising from sequencing error) that are correctly identified or classified as being a variant of interest (e.g., somatic variant or germline variant).

In some cases, the sequencing depth may be adjusted such that a desired specificity of variant calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The specificity of variant calling may be calculated as the percentage of non-variants that are correctly identified or classified as not being a variant of interest (e.g., somatic variant or germline variant).

In some cases, the sequencing depth may be adjusted such that a desired positive predictive value (PPV) of variant calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The PPV of variant calling may be calculated as the percentage of identified or called variants that correspond to a true biological variant.

In some cases, the sequencing depth may be adjusted such that a desired negative predictive value (NPV) of variant calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The NPV of variant calling may be calculated as the percentage of identified or called non-variants that truly correspond to a non-biological variant.

Subjects and Applications

In some cases, the methods, systems, and devices of the present disclosure are applicable to diagnose, prognosticate, or monitor disease progression in a subject. For example, a subject can be a human patient, such as a cancer patient, a patient at risk for cancer, a patient suspected of having cancer, or a patient with a family or personal history of cancer. The sample from the subject can be used to analyze whether or not the subject carries somatic variants that are implicated in certain diseases or conditions, e.g., cancer, Neurofibromatosis 1, McCune-Albright, incontinentia pigmenti, paroxysmal nocturnal hemoglobinuria, Proteus syndrome, or Duchenne Muscular Dystrophy. The sample from the subject can be used to determine whether or not the subject carries somatic variants and can be used to diagnose, prognosticate, or monitor any cancer, e.g., any cancer disclosed herein.

In some cases, diagnosis of somatic variants can provide valuable information for guiding the therapeutic intervention, e.g., for the cancer of the subject. For instance, genetic mutations can directly affect drug tolerance in many cancer types; therefore, understanding the underlying genetic variants can be useful for providing precision medical treatment of a cancer patient. In some cases, the methods, systems, and devices of the present disclosure can be used for application to drug development or developing a companion diagnostic. In some cases, the methods, systems, and devices of the present disclosure can also be used for predicting response to a therapy. In some cases, the methods, systems, and devices of the present disclosure can also be used for monitoring disease progression. In some cases, the methods, systems, and devices of the present disclosure can also be used for detecting relapse of a condition, e.g., cancer. A presence or absence of a known somatic variant or appearance of new somatic variant can be correlated with different stages of disease progression, e.g., different stages of cancers. As cancer progresses from early stage to late stage, an increased number or amount of new mutations can be detected by the methods, systems, or devices of the present disclosure.

The subject may be monitored over time by assessing the disease of the subject at two or more time points (e.g., during a course of treatment, to monitor for signs of disease progression, recurrence, resistance, or relapse, or to determine a definitive diagnosis after an indeterminate assessment). The assessing may be based at least in part on quantitative measures of sequencing reads (e.g., obtained from cell-free samples, tumor tissue samples, and/or normal tissue samples), somatic variants, and/or germline variants of the subject, as determined at each of the two or more time points.

In some cases, a difference in the quantitative measures of sequencing reads, somatic variants, and/or germline variants of the subject determined between the two or more time points may be indicative of one or more clinical indications, such as (i) a diagnosis of a disease of the subject, (ii) a prognosis of a disease of the subject, (iii) an increased risk of a disease of the subject, (iv) a decreased risk of a disease of the subject, (v) an efficacy of a course of treatment for treating a disease of the subject, and (vi) a non-efficacy of a course of treatment for treating a disease of the subject.

In some cases, a difference in the quantitative measures of sequencing reads, somatic variants, and/or germline variants of the subject determined between the two or more time points may be indicative of a diagnosis of the disease of the subject. For example, if the disease was not detected in the subject at an earlier time point but was detected in the subject at a later time point, then the difference is indicative of a diagnosis of the disease of the subject. A clinical action or decision may be made based on this indication of diagnosis of the disease of the subject, such as, for example, prescribing a new therapeutic intervention for the subject. The clinical action or decision may comprise recommending the subject for a secondary clinical test to confirm the diagnosis of the disease. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, a cell-free biological cytology, or any combination thereof.

In some cases, a difference in the quantitative measures of sequencing reads, somatic variants, and/or germline variants of the subject determined between the two or more time points may be indicative of a prognosis of the disease of the subject.

In some cases, a difference in the quantitative measures of sequencing reads, somatic variants, and/or germline variants of the subject determined between the two or more time points may be indicative of an increased risk of the disease of the subject. For example, if the disease was not detected in the subject both at an earlier time point and at a later time point, and if the difference is a positive difference (e.g., the quantitative measures increased from the earlier time point to the later time point), then the difference may be indicative of the subject having an increased risk of the disease. A clinical action or decision may be made based on this indication of the increased risk of the disease, e.g., prescribing a new therapeutic intervention, switching therapeutic interventions (e.g., ending a current treatment and prescribing a new treatment), or adjusting the monitoring schedule for the subject. The clinical action or decision may comprise recommending the subject for a secondary clinical test to confirm the increased risk of the disease. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, or any combination thereof.

In some cases, a difference in the quantitative measures of sequencing reads, somatic variants, and/or germline variants of the subject determined between the two or more time points may be indicative of a decreased risk of the disease of the subject. For example, if the disease was not detected in the subject both at an earlier time point and at a later time point, and if the difference is a negative difference (e.g., the quantitative measures decreased from the earlier time point to the later time point), then the difference may be indicative of the subject having a decreased risk of the disease. A clinical action or decision may be made based on this indication of the decreased risk of the disease, e.g., continuing or ending a current therapeutic intervention or adjusting the monitoring schedule for the subject. The clinical action or decision may comprise recommending the subject for a secondary clinical test to confirm the decreased risk of the disease. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, a cell-free biological cytology, or any combination thereof.

In some cases, a difference in the quantitative measures of sequencing reads, somatic variants, and/or germline variants of the subject determined between the two or more time points may be indicative of an efficacy of the course of treatment for treating the disease of the subject. For example, if the disease was detected in the subject at an earlier time point but was not detected in the subject at a later time point, then the difference may be indicative of an efficacy of the course of treatment for treating the disease of the subject. A clinical action or decision may be made based on this indication of the efficacy of the course of treatment for treating the disease of the subject, e.g., continuing or ending a current therapeutic intervention for the subject. The clinical action or decision may comprise recommending the subject for a secondary clinical test to confirm the efficacy of the course of treatment for treating the disease. This secondary clinical test may comprise an imaging test, a blood test, a computed tomography (CT) scan, a magnetic resonance imaging (MM) scan, an ultrasound scan, a chest X-ray, a positron emission tomography (PET) scan, a PET-CT scan, a cell-free biological cytology, or any combination thereof.

In some cases, a difference in the quantitative measures of sequencing reads, somatic variants, and/or germline variants of the subject determined between the two or more time points may be indicative of a non-efficacy of the course of treatment for treating the disease of the subject (e.g., associated with disease progression, recurrence, resistance, or relapse). For example, if the disease was detected in the subject both at an earlier time point and at a later time point, and if the difference is a positive or zero difference (e.g., the quantitative measures increased or remained at a constant level from the earlier time point to the later time point), and/or if an efficacious treatment was indicated at an earlier time point, then the difference may be indicative of a non-efficacy of the course of treatment for treating the disease of the subject. A clinical action or decision may be made based on this indication of the non-efficacy of the course of treatment for treating the disease of the subject, e.g., prescribing a new therapeutic intervention or switching therapeutic interventions (e.g., ending a current therapeutic intervention and/or switching to (e.g., prescribing) a different new therapeutic intervention) for the subject. The clinical action or decision may comprise recommending the subject for a secondary clinical test to confirm the non-efficacy of the course of treatment for treating the disease. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, or any combination thereof.

In some cases, the subject may be monitored on a schedule at regular intervals, such as every 1, 2, 3, 4, 5, 6, or 7 days, every 1, 2, 3, or 4 weeks, every 1, 2, 3, 4, 5, 6, 7, 8, 9, 10, 11 or 12 months, or every 1, 2, 3, 4, 5, 6, 7, 8, 9, or 10 years. The monitoring schedule may be adjusted based on the monitoring assessments. A report indicative of the monitoring assessments may be generated and sent to the subject or a health care provider of the subject.

Methods, systems, and devices of the present disclosure can be used to analyze biological sample from a subject. The subject can be any human being. The biological sample for somatic variant detection can be obtained from a tissue of interest, e.g., a pathological tissue, e.g., a tumor tissue. Alternatively, the biological sample can be a liquid biological sample containing cell-free nucleic acids, such as blood, plasma, serum, saliva, urine, amniotic fluid, pleural effusion, tears, seminal fluid, peritoneal fluid, and cerebrospinal fluid. Cell-free nucleic acids can comprise cell-free DNA or cell-free RNA. The cell-free nucleic acids used by methods and systems of the present disclosure can be nucleic acid molecules outside of cells in a biological sample. Cell-free DNA can occur naturally in the form of short fragments.

In some cases, the subject is in a particular stage of cancer treatment. In some cases, the subject can have or be suspected of having cancer. In some cases, whether the subject has cancer is unknown. A subject can have any type of cancer or tumor. In some cases, the biological sample is from any type of tumor tissue, including, but not limited to, adrenal cancer, anal cancer, basal cell carcinoma, bile duct cancer, bladder cancer, cancer of the blood, bone cancer, a brain tumor, breast cancer, bronchus cancer, cancer of the cardiovascular system, cervical cancer, colon cancer, colorectal cancer, cancer of the digestive system, cancer of the endocrine system, endometrial cancer, esophageal cancer, eye cancer, gallbladder cancer, a gastrointestinal tumor, hepatocellular carcinoma, kidney cancer, hematopoietic malignancy, laryngeal cancer, leukemia, liver cancer, lung cancer, lymphoma, melanoma, mesothelioma, cancer of the muscular system, Myelodysplastic Syndrome (MDS), myeloma, nasal cavity cancer, nasopharyngeal cancer, cancer of the nervous system, cancer of the lymphatic system, oral cancer, oropharyngeal cancer, osteosarcoma, ovarian cancer, pancreatic cancer, penile cancer, pituitary tumors, prostate cancer, rectal cancer, renal pelvis cancer, cancer of the reproductive system, cancer of the respiratory system, sarcoma, salivary gland cancer, skeletal system cancer, skin cancer, small intestine cancer, stomach cancer, testicular cancer, throat cancer, thymus cancer, thyroid cancer, a tumor, cancer of the urinary system, uterine cancer, vaginal cancer, or vulvar cancer. The lymphoma can be any type of lymphoma including B-cell lymphoma (e.g., diffuse large B-cell lymphoma, follicular lymphoma, small lymphocytic lymphoma, mantle cell lymphoma, marginal zone B-cell lymphoma, Burkitt lymphoma, lymphoplasmacytic lymphoma, hairy cell leukemia, or primary central nervous system lymphoma) or a T-cell lymphoma (e.g., precursor T-lymphoblastic lymphoma, or peripheral T-cell lymphoma). In some cases, the patient is a leukemia patient, and the biological sample is the patient's blood. The leukemia can be any type of leukemia including acute leukemia or chronic leukemia. Types of leukemia include acute myeloid leukemia, chronic myeloid leukemia, acute lymphocytic leukemia, acute undifferentiated leukemia, or chronic lymphocytic leukemia. In some cases, the cancer patient does not have a particular type of cancer. For example, in some instances, the patient can have a cancer that is not breast cancer.

A subject applicable by the methods of the present disclosure can be of any age and can be an adult, infant or child. In some cases, the subject is within any age range (e.g., between 0 and 20 years old, between 20 and 40 years old, or between 40 and 90 years old, or even older). In some cases, the subject as described herein can be a non-human animal, such as non-human primate, pig, dog, cow, sheep, mouse, rat, horse, donkey, or camel.

The reference genome of the subject as described herein can be a reference genome of a human being when the subject is a human, such as hg19, GRChg37, GRChg38, NCBI Build 34, NCBI Build 35, or NCBI Build 36.1. The human reference genome can be the genome of an individual human. The human genome can be a consensus sequence.

Data Processing

Parallel Processing

In an aspect, the present disclosure provides a computer-implemented method for performing parallel processing of a plurality of sequencing reads, comprising: (a) obtaining a sequence alignment map (SAM) or binary alignment map (BAM) file of the plurality of sequencing reads; (b) creating a plurality of sets of the plurality of sequencing reads, wherein each set of the plurality of sets comprises all genomic regions represented in the plurality of sequencing reads that are closer to each other than a pre-determined maximum interval; (c) splitting the SAM or BAM file into a plurality of distinct microSAM or microBAM files, wherein each distinct microSAM or microBAM file comprises a different set from among the plurality of sets of the sequencing reads; and (d) performing parallel processing of the plurality of distinct microSAM or microBAM files to identify genetic variants in the plurality of sequencing reads.

Performing variant calling on large input files corresponding to DNA sequence data can involve significant computational complexity and therefore take a long time to complete. Given that variant calling can involve significant time and computational resources to complete and can be a recurrent process as part of research and development activities, the parallelization of the variant calling process can significantly increase iteration frequency. Input files corresponding to DNA sequence data can be processed in a highly parallelized manner for increased throughput and faster algorithms. The parallelized process can be used with systems and methods of the present disclosure, or any other variant calling method. This can comprise splitting the input files (e.g., SAM, BAM, and/or CRAM) files into small regions to parallelize redundant bioinformatic processing. Such approaches can effectively reduce or minimize the size of the induced microbams and maximizing compatibility with variant calling algorithms. For example, the results can be obtained using a single instance with 96 cores, or a Pachyderm cluster.

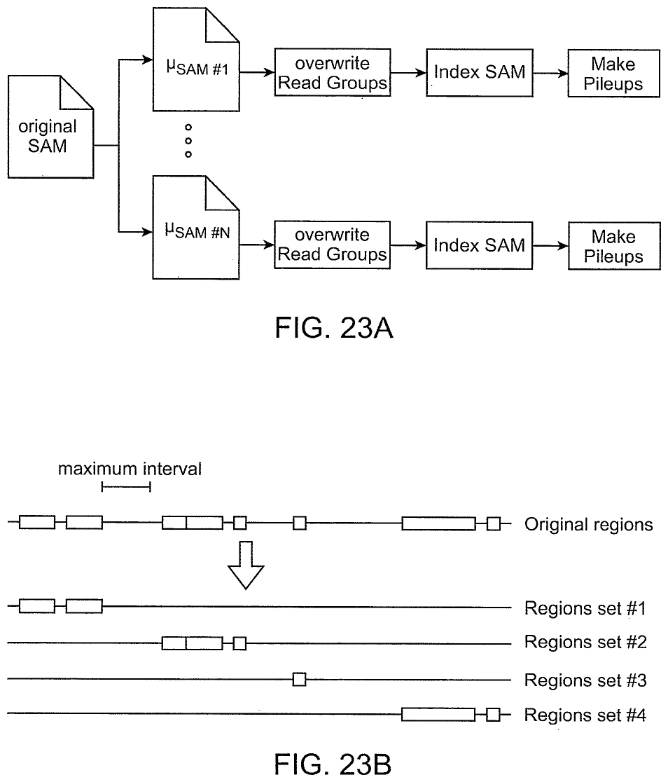

FIG. 23A shows an example of splitting an original SAM file into N different microSAM or microBAM files. The parallelized process can comprise computing the coverage of the input SAM (e.g., using a list of all regions). Next, the parallelized process can comprise defining a maximum interval. For example, the maximum interval may be about 10 base pairs (bp), about 20 bp, about 30 bp, about 40 bp, about 50 bp, about 60 bp, about 70 bp, about 80 bp, about 90 bp, about 100 bp, about 150 bp, about 200 bp, about 250 bp, about 300 bp, about 350 bp, about 400 bp, about 450 bp, about 500 bp, about 600 bp, about 700 bp, about 800 bp, about 900 bp, about 1,000 bp (kbp), about 2 kbp, about 3 kbp, about 4 kbp, about 5 kbp, about 6 kbp, about 7 kbp, about 8 kbp, about 9 kbp, about 10 kbp, about 15 kbp, about 20 kbp, about 25 kbp, about 30 kbp, about 35 kbp, about 40 kbp, about 45 kbp, about 50 kbp, about 60 kbp, about 70 kbp, about 80 kbp, about 90 kbp, about 100 kbp, about 200 kbp, about 300 kbp, about 400 kbp, about 500 kbp, about 600 kbp, about 700 kbp, about 800 kbp, about 900 kbp, about 1000 kbp (1 Mbp), about 2 Mbp, about 3 Mbp, about 4 Mbp, about 5 Mbp, about 6 Mbp, about 7 Mbp, about 8 Mbp, about 9 Mbp, about 10 Mbp, about 20 Mbp, about 30 Mbp, about 40 Mbp, about 50 Mbp, about 60 Mbp, about 70 Mbp, about 80 Mbp, about 90 Mbp, about 100 Mbp, about 200 Mbp, about 300 Mbp, about 400 Mbp, about 500 Mbp, about 600 Mbp, about 700 Mbp, about 800 Mbp, about 900 Mbp, about 1000 Mbp, about 1500 Mbp, about 2000 Mbp, about 2500 Mbp, or about 3000 Mbp. In some embodiments, the maximum interval corresponds to an entire chromosomal region. In some embodiments, the maximum interval may be about 1, 2, 3, 4, 5, 6, 7, 8, 9, 10, 12, 14, 16, 18, 20, 25, 30, 35, 40, 45, 50, 60, 70, 80, 90, 100, 150, 200, 250, 300, 350, 400, 450, 500, 600, 700, 800, 900, 1,000, 2,000, 3,000, 4,000, 5,000, 6,000, 7,000, 8,000, 9,000, or 10,000 regions. Next, the parallelized process can comprise putting in the same set all regions that are closer to each other than the previously defined maximum interval. This operation generates a list of region sets (as shown in FIG. 23B). Next, the parallelized process can comprise splitting or slicing the original SAM into a plurality of different sets, corresponding to the list of region sets. For each of the different sets, a microSAM or microBAM file can be created. Next, the parallelized process can comprise applying computation (e.g., non-parallelized computation or parallelized computation) on the microSAMs, which can include overwriting read groups, indexing the SAM or BAM or microSAM or microBAM files, and generating pileups from the microSAM or microBAM files.

The parallelized process may not be limited by the number of processor (e.g., CPU and/or GPU) cores available in a given machine on which the algorithm is performed, but by the size of the largest set of regions. For the same SAM or BAM file, the computation time can be reduced significantly to about 30 minutes (e.g., resulting in a speedup of about 48× relative to a non-parallelized computation time of 24 hours). In some cases, the parallelization process produces a speedup of at least about 1.1×, at least about 1.2×, at least about 1.3×, at least about 1.4×, at least about 1.5×, at least about 1.6×, at least about 1.7×, at least about 1.8×, at least about 1.9×, at least about 2×, at least about 3×, at least about 4×, at least about 5×, at least about 10×, at least about 15×, at least about 20×, at least about 25×, at least about 30×, at least about 35×, at least about 40×, at least about 45×, at least about 50×, at least about 60×, at least about 70×, at least about 80×, at least about 90×, at least about 100×, or more than about 100×, as compared to other non-parallelized methods of variant calling.

The parallelized process can be performed with a total computation time (e.g., runtime) of no more than about 7 days, no more than about 6 days, no more than about 5 days, no more than about 4 days, no more than about 3 days, no more than about 48 hours, no more than about 36 hours, no more than about 24 hours, no more than about 22 hours, no more than about 20 hours, no more than about 18 hours, no more than about 16 hours, no more than about 14 hours, no more than about 12 hours, no more than about 10 hours, no more than about 9 hours, no more than about 8 hours, no more than about 7 hours, no more than about 6 hours, no more than about 5 hours, no more than about 4 hours, no more than about 3 hours, no more than about 2 hours, no more than about 60 minutes, no more than about 45 minutes, no more than about 30 minutes, no more than about 20 minutes, no more than about 15 minutes, no more than about 10 minutes, or no more than about 5 minutes.

In some cases, the methods and systems of the present disclosure may be performed using a single-core or multi-core machine, such as a dual-core, 3-core, 4-core, 5-core, 6-core, 7-core, 8-core, 9-core, 10-core, 12-core, 14-core, 16-core, 18-core, 20-core, 22-core, 24-core, 26-core, 28-core, 30-core, 32-core, 34-core, 36-core, 38-core, 40-core, 42-core, 44-core, 46-core, 48-core, 50-core, 52-core, 54-core, 56-core, 58-core, 60-core, 62-core, 64-core, 96-core, 128-core, 256-core, 512-core, or 1,024-core machine, or a multi-core machine having more than 1,024 cores. In some cases, the methods and systems of the present disclosure may be performed using a distributed network, such as a cloud computing network, which is configured to provide a similar functionality as a single-core or multi-core machine.

In some cases, the methods and systems of the present disclosure may be performed using an input SAM, BAM, or CRAM file having a size of about 1 Mb, 10 Mb, 50 Mb, 100 Mb, 500 Mb, 1 Gb, 2 Gb, 3 Gb, 4 Gb, 5 Gb, 6 Gb, 7 Gb, 8 Gb, 9 Gb, 10 Gb, 15 Gb, 20 Gb, 25 Gb, 30 Gb, 35 Gb, 40 Gb, 45 Gb, 50 Gb, 55 Gb, 60 Gb, 65 Gb, 70 Gb, 75 Gb, 80 Gb, 85 Gb, 90 Gb, 95 Gb, 100 Gb, 110 Gb, 120 Gb, 130 Gb, 140 Gb, 150 Gb, 160 Gb, 170 Gb, 180 Gb, 190 Gb, 200 Gb, 250 Gb, 300 Gb, 350 Gb, 400 Gb, 450 Gb, 500 Gb, 550 Gb, 600 Gb, 650 Gb, 700 Gb, 750 Gb, 800 Gb, 850 Gb, 900 Gb, 950 Gb, 1000 Gb, or more than about 1000 Gb. In some cases, the runtime may increase linearly with the file size of the dataset that is processed for variant calling. In some cases, the runtime may increase logarithmically with the file size of the dataset that is processed for variant calling.

In some cases, the methods and systems of the present disclosure may be performed using a depth of sequencing of about 1×, 2×, 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, 110×, 120×, 130×, 140×, 150×, 160×, 170×, 180×, 190×, 200×, 250×, 300×, 350×, 400×, 450×, 500×, 550×, 600×, 650×, 700×, 750×, 800×, 850×, 900×, 950×, 1000×, or more than about 1000×. In some cases, the runtime may increase linearly with the depth of sequencing of the dataset that is processed for variant calling. In some cases, the runtime may increase logarithmically with the depth of sequencing of the dataset that is processed for variant calling.

In some cases, the methods and systems of the present disclosure may be performed using a total number of sequencing reads of about 100, 200, 300, 400, 500, 600, 700, 800, 900, 1 thousand, 2 thousand, 3 thousand, 4 thousand, 5 thousand, 6 thousand, 7 thousand, 8 thousand, 9 thousand, 10 thousand, 15 thousand, 20 thousand, 25 thousand, 30 thousand, 35 thousand, 40 thousand, 45 thousand, 50 thousand, 55 thousand, 60 thousand, 65 thousand, 70 thousand, 75 thousand, 80 thousand, 85 thousand, 90 thousand, 95 thousand, 100 thousand, 110 thousand, 120 thousand, 130 thousand, 140 thousand, 150 thousand, 160 thousand, 170 thousand, 180 thousand, 190 thousand, 200 thousand, 250 thousand, 300 thousand, 350 thousand, 400 thousand, 450 thousand, 500 thousand, 550 thousand, 600 thousand, 650 thousand, 700 thousand, 750 thousand, 800 thousand, 850 thousand, 900 thousand, 950 thousand, 1 million, 2 million, 3 million, 4 million, 5 million, 6 million, 7 million, 8 million, 9 million, 10 million, 15 million, 20 million, 25 million, 30 million, 35 million, 40 million, 45 million, 50 million, 55 million, 60 million, 65 million, 70 million, 75 million, 80 million, 85 million, 90 million, 95 million, 100 million, 110 million, 120 million, 130 million, 140 million, 150 million, 160 million, 170 million, 180 million, 190 million, 200 million, 250 million, 300 million, 350 million, 400 million, 450 million, 500 million, 550 million, 600 million, 650 million, 700 million, 750 million, 800 million, 850 million, 900 million, 950 million, 1 billion, 2 billion, 3 billion, 4 billion, 5 billion, 6 billion, 7 billion, 8 billion, 9 billion, 10 billion, 15 billion, 20 billion, 25 billion, 30 billion, 35 billion, 40 billion, 45 billion, 50 billion, 55 billion, 60 billion, 65 billion, 70 billion, 75 billion, 80 billion, 85 billion, 90 billion, 95 billion, 100 billion, 110 billion, 120 billion, 130 billion, 140 billion, 150 billion, 160 billion, 170 billion, 180 billion, 190 billion, 200 billion, 250 billion, 300 billion, 350 billion, 400 billion, 450 billion, 500 billion, 550 billion, 600 billion, 650 billion, 700 billion, 750 billion, 800 billion, 850 billion, 900 billion, 950 billion, or 1 trillion sequencing reads. In some cases, the runtime may increase linearly with the number of total number of sequencing reads of the dataset that is processed for variant calling. In some cases, the runtime may increase logarithmically with the number of total number of sequencing reads of the dataset that is processed for variant calling.

In some cases, for a given sequencing depth and a given computational power, the runtime may increase linearly with the number of sequencing reads that are processed. For example, on an 8-core machine, a 60 Gb SAM file of 150× depth with 926,000,000 sequencing reads is processed by Mutect with a total runtime of about 35 hours. In comparison, using the parallelization process with methods and systems of the present disclosure, the same SAM file was processed with a total runtime of about 5 hours. Therefore, using methods and systems of the present disclosure, the parallelization process produced a speedup of about 7× was achieved as compared to performing the same process using Mutect.

In some cases, the method further comprises determining a coverage of the SAM or BAM file. In some cases, (d) comprises one or more of: overwriting read groups, indexing the SAM or BAM file or the plurality of distinct microSAM or microBAM files, and generating pileups from the plurality of distinct microSAM or microBAM files. In some cases, (d) comprises identifying somatic genetic variants in the plurality of sequencing reads, germline genetic variants in the plurality of sequencing reads, false-positive genetic variants arising from sequencing error, or a combination thereof.

Pileup Matrix

In another aspect, the present disclosure provides a computer-implemented method for generating a pileup matrix, comprising: (a) obtaining a plurality of sequencing reads of nucleic acids (e.g., cell-free nucleic acids, nucleic acids from a tumor tissue, nucleic acids from a tissue suspected of containing a tumor or genetic variants (e.g., somatic variants or germline variants)) from a subject and a plurality of sequencing reads of nucleic acids from a normal tissue of the subject; (b) creating a pileup image from the plurality of sequencing reads of the cell-free nucleic acids; (c) creating a normal pileup image from the plurality of sequencing reads of the nucleic acids from the normal tissue; (d) applying a coloration algorithm to the pileup image and the normal pileup image; and (e) concatenating the pileup image and the normal pileup image, thereby generating the pileup matrix.

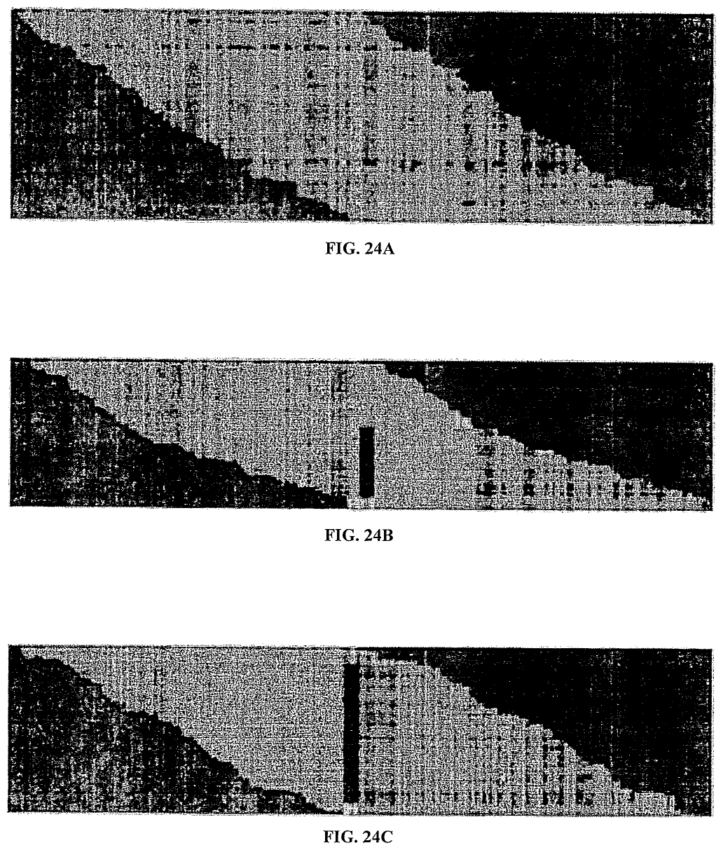

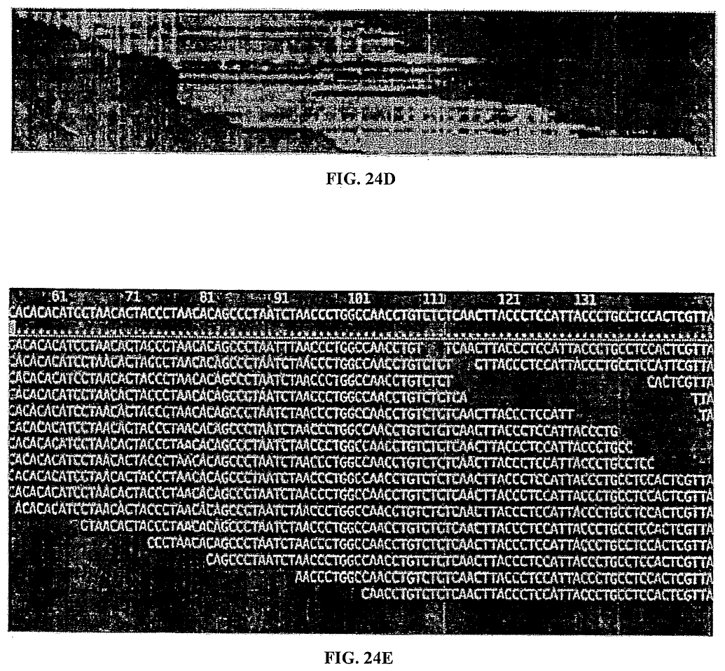

In some cases, pileup images generated from DNA sequencing read data can be difficult to represent in such a manner as to be efficiently interpreted by machine learning algorithms (e.g., models) and/or humans. For example, FIGS. 24A-24E show examples of pileup images that can be challenging for humans to visually interpret. In order to generate a compact, readable, yet complete representation of genetic variants, such as somatic variants, a set of procedures or rules can be performed to process such pileup images (e.g., which can include complex mutations) to make them both more compact in size and more readable and visually interpretable. Such approaches can have advantages, including faster generation and training, enhanced performance of many deep learning models (e.g., classifiers), and easier and faster debugging of machine learning models. The generation of pileup images can be used with systems and methods of the present disclosure, or any other variant calling method.

First, the data can be loaded, including a test sample BAM file, a normal BAM file (e.g., generated by sequencing a normal tissue sample from a subject), and a list of candidates (e.g., genomic positions where there may be somatic variants (such as single nucleotide polymorphisms (SNPs), germline mutations, sequencing noise, or PCR noise). In some cases, the test sample BAM file is generated by sequencing a test sample, such as a tumor tissue sample, a cell-free sample, a bodily fluid sample, or a tissue suspected of containing a tumor or genetic variants (e.g., somatic variants or germline variants) from a subject.

Next, a test sample (e.g., tumor) image can be created by obtaining a set of pileup sequencing reads from the tumor BAM file, applying a Concise Idiosyncratic Gapped Alignment Report (CIGAR) correction, applying a coloration algorithm, applying a quality filter, and adding a reference. In some cases, obtaining the set of pileup sequencing reads comprises, for a given location use of ‘pysam’, querying the ordered reads that share the location. In some cases, applying the CIGAR correction comprises: in the case of 0 (e.g., a match), keeping the base call as is; in the case of 1 (e.g., an insertion or INS), removing all inserted bases and replacing them by one base of a specific color for insertions; and in the case of 2 (e.g., a deletion or DEL), applying a right offset of the size of the deletion. In some cases, applying the coloration algorithm comprises assigning each base to a given color found by categorically combining the three RGB color channels (red, green, and blue). In some cases, applying the quality filter comprises, for each base, multiplying the color of the base by the quality score (e.g., between 0 and 1), thereby applying a desaturation filter. In some cases, adding the reference comprises following the same coloration process, but with a constant quality of 1, to create and add a reference row to the top.

Next, a normal image can be created by obtaining a set of pileup sequencing reads from the normal BAM file, and processing the pileup sequencing reads from the normal BAM file in a similar manner as for the pileup sequencing reads from the tumor BAM file.

Finally, the tumor and normal images can be concatenated. This can be performed by concatenating on the second axis (horizontal) both the tumor and normal matrices, thereby generating a pileup matrix.

In some cases, creating the pileup image or the normal pileup image comprises applying a Concise Idiosyncratic Gapped Alignment Report (CIGAR) correction. In some cases, the method further comprises applying a quality filter to the pileup image and the normal pileup image. In some cases, the method further comprises adding a reference to the pileup image and the normal pileup image. In some cases, the method further comprises processing the pileup matrix to identify genetic variants in the plurality of sequencing reads of the test sample nucleic acids (e.g., obtained from samples such as a tumor tissue sample, a cell-free sample, a bodily fluid sample, or a tissue suspected of containing a tumor or genetic variants (e.g., somatic variants or germline variants)) or the plurality of sequencing reads of the nucleic acids from the normal tissue.

Computer System

Any of the methods disclosed herein can be performed and/or controlled by one or more computer systems or devices. In some examples, any operation of the methods disclosed herein can be wholly, individually, or sequentially performed and/or controlled by one or more computer systems. Any of the computer systems mentioned herein can utilize any suitable number of subsystems. In some instances, a computer system includes a single computer apparatus, where the subsystems can be the components of the computer apparatus. In other cases, a computer system can include multiple computer apparatuses, each being a subsystem, with internal components. A computer system can include desktop and laptop computers, tablets, mobile phones and other mobile devices.

The subsystems can be interconnected via a system bus. Additional subsystems include a printer, keyboard, storage device(s), and monitor that can be coupled to display adapter. Peripherals and input/output (I/O) devices, which couple to I/O controller, can be connected to the computer system by any number of connections, such as an input/output (I/O) port (e.g., USB, FireWire®). For example, an I/O port or external interface (e.g., Ethernet, Wi-Fi, etc.) can be used to connect computer system to a wide area network such as the Internet, a mouse input device, or a scanner. The interconnection via system bus can allow the central processor to communicate with each subsystem and to control the execution of a plurality of instructions from system memory or the storage device(s) (e.g., a fixed disk, such as a hard drive, or optical disk), as well as the exchange of information between subsystems. The system memory and/or the storage device(s) can embody a computer readable medium. Another subsystem can be a data collection device, such as a camera, microphone, accelerometer, and the like. Any of the data mentioned herein can be output from one component to another component and can be output to the user.

A computer system can include a plurality of the same components or subsystems, e.g., connected together by external interface or by an internal interface. In some cases, computer systems, subsystem, or apparatuses can communicate over a network. In such instances, one computer can be considered a client and another computer a server, where each can be part of a same computer system. A client and a server can each include multiple systems, subsystems, or components.

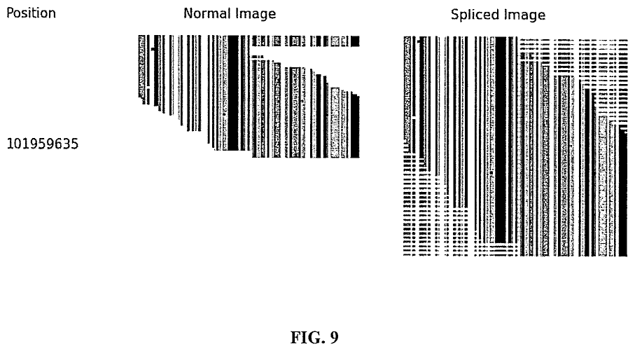

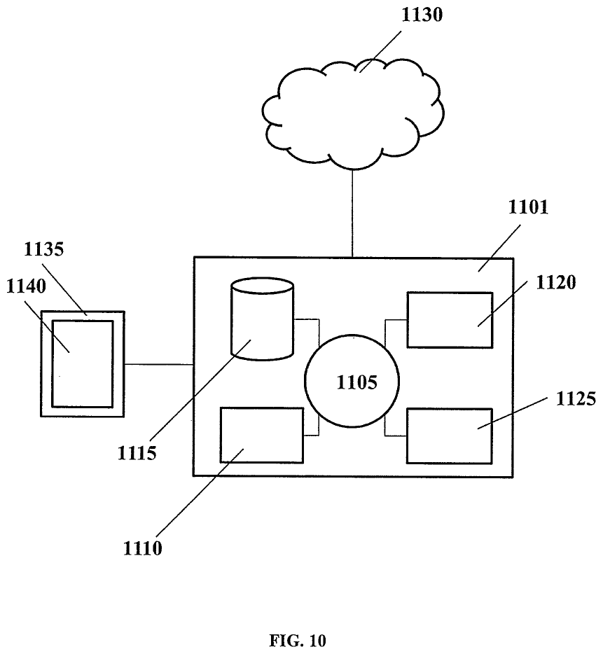

The present disclosure provides computer systems that are programmed to implement methods of the disclosure. FIG. 10 shows a computer system 1101 that can be programmed or otherwise configured to detect a nucleotide variant, e.g., germline or somatic variant, in sequencing reads. The computer system 1101 can implement and/or regulate various aspects of the methods provided in the present disclosure, such as, for example, controlling sequencing of the nucleic acid molecules from a biological sample, performing various operations of the bioinformatics analyses of sequencing data as described herein, integrating data collection, analysis and result reporting, and data management. The computer system 1101 can be an electronic device of a user or a computer system that can be remotely located with respect to the electronic device. The electronic device can be a mobile electronic device.

The computer system 1101 can include a central processing unit (CPU, also “processor” and “computer processor” herein) 1105, which can be a single core or multi core processor, or a plurality of processors for parallel processing. The computer system 1101 can also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 can be in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that can be in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions can be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that can be in communication with the computer system 1101 through an intranet or the Internet.

The computer system 1101 can communicate with one or more remote computer systems through the network 1130. For instance, the computer system 1101 can communicate with a remote computer system of a user (e.g., a Smart phone installed with application that receives and displays results of sample analysis sent from the computer system 1101). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods of the present disclosure, such as the computer system 1101, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media can be involved in carrying one or more sequences of one or more instructions to a processor for execution.

The computer system 1101 can include or be in communication with an electronic display 1135 that comprises a user interface (UI) 1140 for providing, for example, results of sample analysis, such as, but not limited to graphic showings of genomic locations of somatic variants, types and frequencies of somatic variants, time course analyses of somatic variants, diagnosis of cancer, and prediction of drug responses. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, control sequencing of the nucleic acid molecules from a sample, direct collection of sequencing data, analyzing the sequencing data, or determining a classification of pathology based on the analyses of the sequencing data.

In some cases, as shown in FIG. 11, a sample 1202 can be obtained from a subject 1201, such as a human subject. A sample 1202 can be subjected to one or more methods as described herein, such as sequencing and bioinformatic analysis of the sequencing reads. One or more results from a method can be input into a processor 1204. One or more input parameters such as a sample identification, subject identification, sample type, a reference, or other information can be input into a processor 1204. One or more metrics from an assay can be input into a processor 1204 such that the processor may produce a result, such as a classification of pathology (e.g., diagnosis) or a recommendation for a treatment. A processor can send a result, an input parameter, a metric, a reference, or any combination thereof to a display 1205, such as a visual display or graphical user interface. A processor 1204 can (i) send a result, an input parameter, a metric, or any combination thereof to a server 1207, (ii) receive a result, an input parameter, a metric, or any combination thereof from a server 1207, (iii) or a combination thereof.

Aspects of the present disclosure can be implemented in the form of control logic using hardware (e.g., an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the presented disclosure, other suitable methods can be used to implement embodiments described herein using hardware, software, or a combination of hardware and software.

Any of the software components or functions described in this application can be implemented as software code to be executed by a processor using any suitable computer language such as, for example, Java, C, C++, C#, Objective-C, Swift, or scripting language such as Perl or Python using, for example, conventional or object-oriented techniques. The software code can be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium can be any combination of such storage or transmission devices.

Such programs can also be encoded and transmitted using carrier signals adapted for transmission via wired, optical, and/or wireless networks conforming to a variety of protocols, including the Internet. As such, a computer readable medium can be created using a data signal encoded with such programs. Computer readable media encoded with the program code can be packaged with a compatible device or provided separately from other devices (e.g., via Internet download). Any such computer readable medium can reside on or within a single computer product (e.g., a hard drive, a CD, or an entire computer system), and can be present on or within different computer products within a system or network. A computer system can include a monitor, printer, or other suitable display for providing any of the results mentioned herein to a user.

Any of the methods described herein can be totally or partially performed with a computer system including one or more processors, which can be configured to perform the operations disclosed herein. Thus, embodiments can be directed to computer systems configured to perform the operations of any of the methods described herein, with different components performing a respective operation or a respective group of operations. Although presented as numbered operations, the operations of the methods disclosed herein can be performed at a same time or in a different order. Additionally, portions of these operations can be used with portions of other operations from other methods. Also, all or portions of an operation can be optional. Additionally, any of the operations of any of the methods can be performed with modules, units, circuits, or other approaches for performing these operations.

### EXAMPLES

The examples below further illustrate the described embodiments without limiting the scope of this disclosure.

### Example 1

### GermlineNET Training and Evaluation

In this example, an example of an implementation of the method disclosed herein, GermlineNET, was constructed, trained, and evaluated.

GermlineNET was constructed as discussed above. For training, Genome In A Bottle (GIAB) was used, which publicly provides a BAM file of the sequenced germline DNA, a FASTA file of the reference genome, and a VCF file of the sites which, with very high confidence, are germline variants. The locations of germline variants provided by the VCF file were used as the ground truth (e.g., true positives). To determine the true negative sites, the same threshold were used as when determining the candidate sites and only selected for those which were are not marked as true positives.

A balanced dataset was used, comprising 48000 images of SNPs, and 48000 images of candidate sites which were not SNPs. They were split by chromosome into a training set and a validation set, with the validation set used for early stopping. Chromosomes 1 to 17 were used for training, and chromosomes 18 to 22 and Chromosome X were used for validation. This gives a total of 72,000 images for training (36,000 images of SNPs and 36,000 images of non-SNPs) and 24,000 images for validation (12,000 images of SNPs and 12,000 images of non-SNPs).

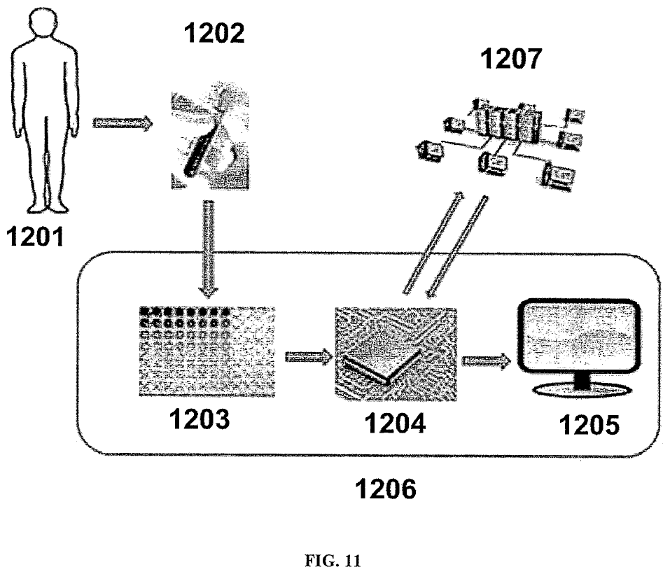

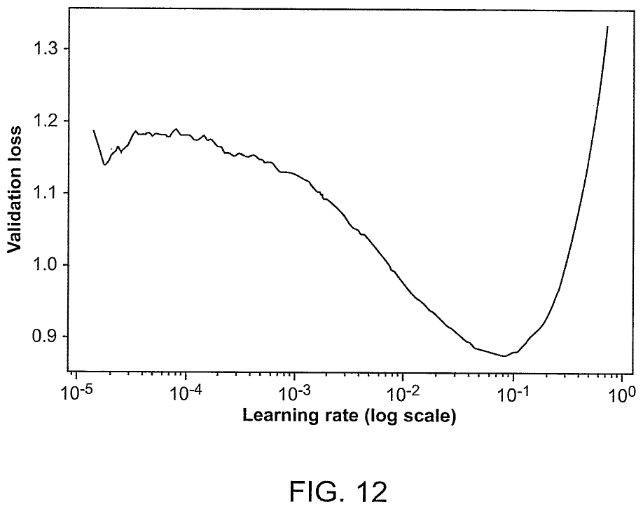

Next, with the data labelled with the ground truth, the model, and a loss function, different hyper-parameters (the model architectures) were used to train the parameters using SGD. The optimal initial learning rate was determined using a method described by, for example, Smith (“Cyclical Learning Rates for Training Neural Networks,” 2015; URL: arxiv.org/abs/1506.01186, which is incorporated by reference herein in its entirety). As shown by FIG. 12, by linearly increasing the learning rate, the loss decreased up to about 10⁻³. The initial learning rate was set to 10⁻⁴, one magnitude higher.

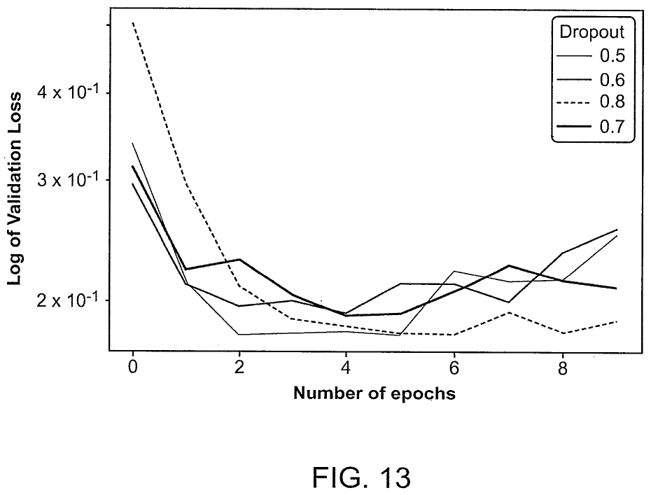

For ResNet34, dropout was introduced to prevent over-fitting. To determine the dropout percentage to use, the number of epochs was plotted against the log of the validation loss, as shown in FIG. 13. At a dropout of 0.9, the model was unable to learn, e.g., the validation did not change. The final dropout percentage was chosen to be 0.8. The Inception architecture did not over-fit, and therefore no regularization was introduced.

To evaluate the GermlineNET's neural network and the effect of different model architectures, the quality layer, and the spliced kernel, a K-fold cross-validation was used. K-fold cross-validation involves partitioning the sample into K equally sized folds (4 folds were used), selecting one fold to use test on, and training the model on the remaining K−1 folds. This cycle was repeated K times, such that each fold was used once as the test set. After each epoch, for the 20 epochs of training, the model was tested on the test fold, with the accuracy, F1 score, receiver operating characteristic area under the curve (ROC AUC), recall and precision all recorded. F1 score was used to select the best model for that training cycle. The metrics from these 4 models (the best model of the 4 training cycles) was then averaged.

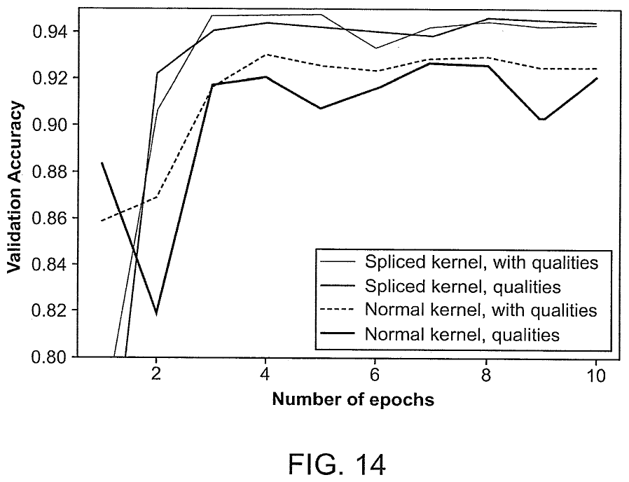

The results are shown in FIG. 14. First, the effect of model architecture is illustrated: ResNet34 resulted in a higher F1 score, AUC, recall and precision than using the Inception v4 architecture. Without the quality layer or spliced kernel, ResNet34 had an F1 score of 93.7% and an accuracy of 93.7%, however, Inception v4 had an F1 score of 81.8% and an accuracy of 76.8%. This effect was consistent without and with the quality layer and spliced kernel. The Inception model uses a more complex architecture, which can use more data to reach the same level of accuracy. Additionally, it uses a variety of different kernel sizes and layers which might have interfered with its ability to learn from the spliced image.

TABLE 2
Results of K-fold cross-validation for GermlineNET's neural network

| Model | Spliced | Quality | Accuracy | F1 | ROC AUC | Recall | Precision |
|---|---|---|---|---|---|---|---|
| ResNet34 | False | False | 93.7 | 93.7 | 98.2 | 94.2 | 93.3 |
| | | True | 94.0 | 94.1 | 98.6 | 94.8 | 93.6 |
| | True | False | 95.3 | 95.3 | 98.9 | 95.7 | 94.9 |
| | | True | 95.3 | 95.4 | 99.0 | 95.6 | 95.2 |
| Inception v4 | False | False | 76.8 | 81.8 | 82.0 | 93.7 | 74.7 |
| | | True | 85.7 | 85.7 | 92.7 | 86.0 | 85.6 |
| | True | False | 67.9 | 71.8 | 74.6 | 79.2 | 67.7 |
| | | True | 81.6 | 81.4 | 88.8 | 81.3 | 81.9 |

Secondly, the observation on the effect of the spliced kernel can be noted. Without the quality scores, the addition of the kernel improved the performance: the accuracy from 93.7% to 95.3% and the F1 score from 93.7% to 95.3%. This effect on the ResNet-based model was similarly seen when the quality score layer was included. However, for the Inception architecture the performance decreased. Additionally, shown in FIG. 14, with the spliced kernel, the ResNet34-based model reached a higher accuracy in a fewer number of epochs.

Thirdly, the addition of the quality layer made an improvement to the performance of the ResNet34-based model, and actually reduced the performance of the Inception4-based model, with and without the spliced kernel. Qualitatively, the limited effect of the quality scores layer is shown in FIG. 14, where there is little observable difference between the validation accuracies of ResNet34 of training on images with and without the quality scores layer.

A general trend can be that recall is higher than precision in the majority of combinations of model, splicing and accuracy. This effect was more pronounced in the Inception-based model than the ResNet34 model.

In summary, different model architectures and the addition of the spliced kernel were tested with GermlineNET using K-fold cross-validation (K=4). Test statistics showed the average of those from the best model of each cross-validation cycle, using F1 score for model selection (Table 1). 20 epochs of training were conducted in each training cycle and statistics were calculated on the test fold. The model architecture ResNet34 resulted in the highest F1 score (93.7%), and using the spliced kernel and adding a quality channel improved this further to 95.3% (FIG. 14).

### Example 2

### SomaticNET Training and Evaluation

In this example, an example of an implementation of the method disclosed herein, SomaticNET, was constructed, trained and evaluated.

SomaticNET was constructed as discussed above. For training SomaticNET, a publicly available semi-simulated mutation data (Meng and Chen, DATABASE “A database of simulated tumor genomes towards ac-curate detection of somatic small variants in cancer.” doi: 10.1101/261503. URL dx.doi.org/10.1101/261503, which is incorporated by reference herein in its entirety) was used. In total, all 22,875 somatic mutations were used as true positives for training, and an equal number of sites without somatic mutations but which met the VAF threshold to act as true negatives were selected. A balanced dataset of 22,875 images of SNVs and 22,875 images of candidate sites which were not SNVs was used. They were split by chromosome into a training set and a validation set, with the validation set used for early stopping. Chromosomes 1 to 17 were used for training, and chromosomes 18 to 22 and Chromosome X were used for validation. This gives a total of 40,638 images for training and 5,112 images for validation.

The Siamese CNN was validated for use as a binary classifier on a verification task: to determine whether two handwritten characters, selected from the Omniglot dataset, are (0) the same, or (1) different. The Omniglot dataset contains 1623 characters from 50 different alphabets, each handwritten by 20 different people. Each character can be used as a different class. Therefore, it can be considered a wide but shallow dataset—there are many classes each with only 20 samples. The dataset was split into 40 training alphabets and 10 validation alphabets.

The task was to determine whether the two images are of the same character or different characters. There was a 50% chance of them being the same character and 50% chance of them being a different character, as illustrated by the example shown in FIG. 15. Training was performed using mini-batch SGD with the gradients calculated through backpropagation. The best-trained model had a validation accuracy of 87.4%. This demonstrates that the Siamese CNN can be functional at image verification tasks.

With the implemented model and loss function, different model architectures and hyper-parameters were evaluated, using mini-batch SGD for training. The optimal learning rate was determined using the learning rate finder described by Smith (Smith, “Cyclical Learning Rates for Training Neural Networks,” 6 2015, URL arxiv.org/abs/1506.01186, which is incorporated by reference herein in its entirety). During training, the training loss and validation loss were plotted against the epoch number. Early stopping was used to select the optimal model.

TABLE 3
Results of K-fold cross-validation for SomaticNET's neural network

| Model | Spliced | Quality | Accuracy | F1 | ROC AUC | Recall | Precision |
|---|---|---|---|---|---|---|---|
| Resnet34 | False | False | 92.5 | 92.2 | 96.2 | 89.6 | 95.4 |
| | | True | 91.5 | 90.9 | 96.8 | 86.6 | 96.2 |
| | True | False | 93.7 | 93.6 | 95.9 | 93.1 | 94.5 |
| | | True | 92.1 | 91.6 | 97.1 | 87.8 | 96.3 |
| Inception v4 | False | False | 51.6 | 44.0 | 52.2 | 40.7 | 52.7 |
| | | True | 51.9 | 27.8 | 52.2 | 20.1 | 55.2 |
| | True | False | 51.8 | 32.9 | 53.2 | 26.5 | 55.3 |
| | | True | 51.4 | 26.8 | 53.2 | 18.8 | 54.0 |

SomaticNET's neural network was evaluated through K-fold cross-validation, with 4 folds, on the semi-simulated tumor data. For each combination of the model architecture, spliced kernel, and quality scores were included, and the model with the highest F1 score was selected and averaged together. The results are shown in Table 3.

The Siamese Network using ResNet34 with and without the quality layers and spliced kernel achieved an F1 score of more than 90% and an accuracy of more than 91%. However, when using the Inception v4-based model, no combinations of quality layers or splicing managed to learn, which resulted in an F1 score below 41.3% and an accuracy below 51.8%, where 50% accuracy is expected to have occurred due to chance.

Using the spliced kernel resulted in an increase in performance both with and without the quality layers for the ResNet34-based model. Without the quality layers, the F1 score increased from 92.2% to 93.6% and the accuracy increased from 92.5% to 93.7%. However, the quality layer resulted in a decrease in performance with and without the spliced kernel.

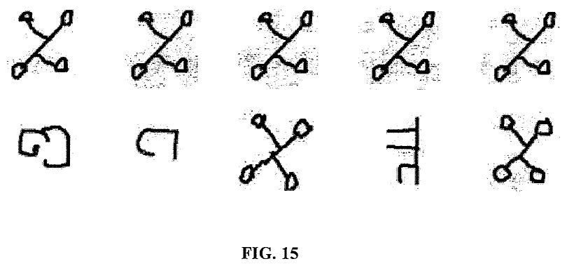

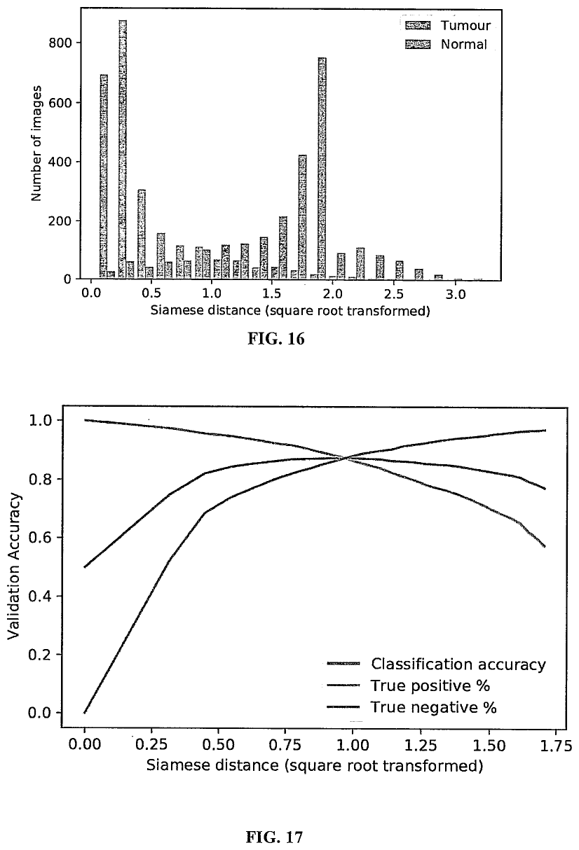

The distribution of Siamese distance on validation data demonstrated that SomaticNET was able to distinguish between tumor and normal tissue classes (FIG. 16). The actual threshold of Siamese distance used for somatic variant calling can be optimized through a grid search, with high validation accuracy, sensitivity, and precision (FIG. 17).

Some methods of evaluating somatic variant callers rely on evaluating a correlation with existing callers on real tumor data. The candidate threshold identified all the sites which were classified as mutations by Mutect and Strelka; however, applying the neural network to this data showed little correlation with these two tools.

### Example 3

### Training Dataset for Somatic Variant Caller

This example shows an example of a labeled dataset, Kappa data. Kappa data can be built to follow true likelihood function of read error occurrence and mutation occurrence by combining two samples (sample A and sample B) which both have high confidence SNP calls (such as the GIAB dataset used in germline calling) together. In-silico downsampling of Sample B to a known concentration can simulate VAF. The downsampling can be possible using Universal Molecular Identifiers (UMIs) which enabled the individual DNA fragments to be tagged and uniquely distinguished and identified, similar to barcodes. Sequencing runs are known to produce vastly different results, however, as both samples are re-sequenced in the same run, the neural network may not be able to cheat. Sample A can then be re-sequenced to provide the “normal” sample. This method can simulate a tumor at a chosen VAF, and the reads are still representative of their corresponding fragment bases. This can enable the sequencing read errors to follow their true likelihood function.

### Example 4

### VaRNN—A Machine Learning Approach for Identifying Somatic Mutations in Cancer

Genomic profiles underlying individual tumors can be identified toward informing personalized cancer treatment strategies, e.g., in the field of precision oncology. This approach can involve performing next generation sequencing (NGS) of a tumor sample and the subsequent identification of genomic aberrations, such as somatic mutations, and solving a classification-style problem to provide clinically useful information, such as potential candidates of targeted therapy. Some approaches of differentiating somatic (tumor) mutations from germline (heritable) variants and sequencing error can be performed using methods such as Bayesian statistics. However, the sensitivity and precision of such methods can be less than ideal. To address this unmet need, using methods and systems of the present disclosure, a bi-directional LSTM (BiLSTM) approach was developed which outperforms other methods. This approach can be incorporated into tumor assessment algorithms (e.g., used by oncologists, physicians, or other clinical health providers of a patient having cancer) to provide statistical confidence in precision oncology treatment choices.

In some cases, the BiLSTM approach produces a speedup of at least about 1.1×, at least about 1.2×, at least about 1.3×, at least about 1.4×, at least about 1.5×, at least about 1.6×, at least about 1.7×, at least about 1.8×, at least about 1.9×, at least about 2×, at least about 3×, at least about 4×, at least about 5×, at least about 10×, at least about 15×, at least about 20×, at least about 25×, at least about 30×, at least about 35×, at least about 40×, at least about 45×, at least about 50×, at least about 60×, at least about 70×, at least about 80×, at least about 90×, at least about 100×, or more than about 100×, as compared to other methods of variant calling.

Cancer is a group of diseases that result from specific changes in the genetic code of otherwise healthy cells. These changes can manifest in a variety of variants or mutations, such as DNA point mutations, insertion or deletion (e.g., indel) mutations, methylation aberrations, copy number aberrations, and structural variants. Next-generation DNA sequencing (NGS) can be used in clinical practice to identify genetic variants, with oncologists using this information for, among other things, determining the best targeted and personalized treatment strategy for patients.

However, variants can be difficult to interpret due to challenges in performing deconvolution of or distinguishing somatic mutations from germline variants, and sequencing error. As a tumor can be the product of an accumulation of mutations over time, mutations can be found in anywhere from 0% to 100% of the sequencing reads in a tumor, and can be difficult to distinguish from germline variant frequencies and false-positive variants arising due to error rate from the sequencing process. Using methods and systems of the present disclosure, machine learning approaches are developed to classify somatic mutations from germline mutations or false-positive variants arising from sequencing error, which can achieve higher accuracy and performance as compared to other methods.

VaRNN refers to a BiLSTM RNN which can classify through cross-image comparisons of tumor and normal tissue sequencing data at potential variant sites within the genome. VaRNN can be trained on both real-world tumor-normal pairs as well as simulated data from a wide range of resources and sequencing techniques. Results demonstrate that VaRNN can outperform some types of Bayesian and machine learning-based somatic variant callers. Data transformation was performed as follows. The initial data used was obtained from the simulated dataset described by Meng and Chen, “A database of simulated tumor genomes towards accurate detection of somatic small variants in cancer,” PloS one, 13(8):e0202982, 2018; which is incorporated by reference herein in its entirety. These include simulated mutations generated through the Bamsurgeon tool. This dataset comprises a well-balanced set of variants, including those that are easy to detect accurately and others that are difficult to detect accurately. A second real-world dataset was obtained from the Multi-Center Mutation Calling in Multiple Cancers (MC3) of the Genomic Data Commons (GDC) (Table 4). These data were re-sequenced in technical replicates across multiple cancer centers to establish a ground truth for borderline variants, thus making sensitivity to mutation calls challenging and difficult.

TABLE 4
Overview of TCGA patients from the MC3 study used when full genome analysis were performed. The
depth can be measured as the average depth of aligned sequencing reads over the coverage area.

| Patient UUID | TCGA Study | Disease type | Primary site | Normal depth | tumor depth |
|---|---|---|---|---|---|
| TCGA-2G-AAGS | TCGA-TGCT | Germ Cell NP¹ | Testis | 99.7882 | 89.226 |
| TCGA-5C-AAPD | TCGA-LIHC | Adenomas and AC² | Liver and IBD³ | 84.0647 | 79.9675 |
| TCGA-AR-A0TT | TCGA-BRCA | Ductal and Lobular NP¹ | Breast | 105.195 | 100.403 |
| TCGA-D6-6827 | TCGA-HNSC | Squamous Cell NP¹ | Lip | 81.4128 | 84.6085 |
| TCGA-FD-A62N | TCGA-BLCA | TCPAP⁴ and CAR⁵ | Bladder | 108.527 | 84.8621 |
| TCGA-KL-8346 | TCGA-KICH | Adenomas and AC² | Kidney | 80.8283 | 84.7701 |
| TCGA-RD-A8N1 | TCGA-STAD | Adenomas and AC² | Stomach | 61.7789 | 60.7841 |
| TCGA-VD-AA8Q | TCGA-UVM | Nevi and Melanomas | Eye and adnexa | 79.1714 | 66.3934 |

¹NP: Neoplasms
²AC: Adenocarcinomas
³IBD: intrahepatic bile ducts

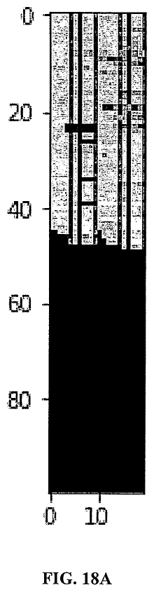

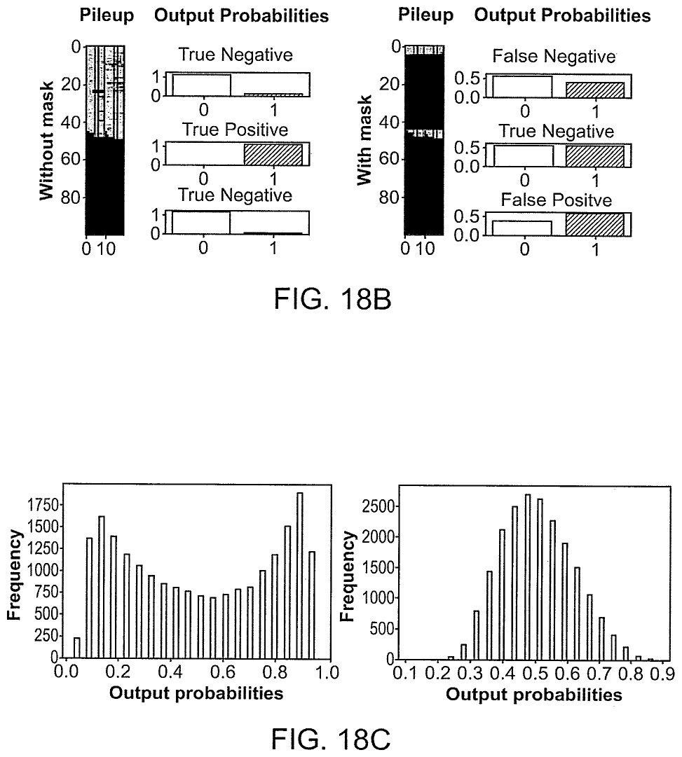

These data were stored in a “BAM” file format, comprising compressed tab-separated text tables containing DNA sequencing reads aligned to regions of the genome. To process these input BAM files using the BiLSTM model, the data was transformed into red-green-blue (RGB) images, which also allows for easy visual inspection of variants. Given the four bases present in DNA (“A”, “T”, “C”, and “G”), fixed values are chosen for each of the RGB channels such that each base has a corresponding color. These values are then modified by the alpha channel according to the base quality score. In this way, the sequencing reads for a given locus are represented in the form of a matrix (as shown in FIG. 18A). This approach represents the input sequencing data in a way that effectively achieves both human readability and machine readability.

$$pair = [ \begin{matrix}{N}_{11} & … & {N}_{1 w} \\ ⋮ & ⋱ & ⋮ \\ {N}_{d 1} & … & {N}_{dw}\end{matrix} \vert \begin{matrix}{T}_{11} & … & {T}_{1 w} \\ ⋮ & ⋱ & ⋮ \\ {T}_{d 1} & … & {T}_{dw}\end{matrix} ]$$

d: the depth
w: the width or number of observed loci
Nᵢⱼ: The normal base at position i and depth d
Tᵢⱼ: The tumor base at position i and depth d

FIG. 18A shows a visual representation of the pair matrix with w=10 and d=10.

The machine learning model was generated as follows. Some approaches to using deep convolutional neural networks (CNNs) toward variant calling in NGS data can be limited by the form of NGS data, which can vary drastically in depth (e.g., a number of sequencing reads at a given locus), both across a plurality of genomic loci within a sequenced sample and between samples. As the depth increases, the size of the corresponding image increases accordingly, which can result in a high computational complexity.

In order to mitigate this issue, LSTM cells were used, which can handle a wide variety of depths, without discarding data, by averaging the values for each locus. To avoid the model giving greater weight to sequencing reads at the bottom of the generated image, BiLSTM cells were used. The model architecture was selected using Hyperopt to find a combination of model size, activations, and cells that provided good performance. Training was performed across all aforementioned datasets. The optimizer Adam, a binary cross entropy loss, and a cyclical learning rate scheduler were used. A variety of resampling strategies were also used to account for large class imbalance inherent to somatic variant data.

Bayesian filtering was performed as follows. To pre-filter the workload for VaRNN, DNA sequencing datasets from both the tumor and normal samples were processed using Freebayes. This was implemented separately for each chromosome using the “−r” flag, and recombined using “bcftools concat.” Candidates were identified as those with a variant allele fraction (VAF)>=0.01 and at least 1 supporting sequencing read in the tumor sample. Those also present at a VAF of at least 0.3 and having at least 2 supporting sequencing reads in the normal sample were removed.

The performance of VaRNN was compared against a plurality of other somatic variant callers, including, for example, MuTect2, SomaticSniper, MuSE, and Neusomatic. Mutect2, SomaticSniper, and MuSE use a Bayesian approach to somatic variant calling, while Neusomatic is a CNN-based method trained on both simulated and real-world data. For MC3 data, the results of these statistical variant callers were available.

MuTect2 was performed by using the following parameters: Docker at broadinstutute/gatk:4.0.5.1; preprocessing using picard AddOrReplaceReadGroups; no runtime specifics; and postprocessing using MuTect2 filtering. SomaticSniper was performed by using the following parameters: Docker at opengenomics/somatic-sniper:latest; no preprocessing; runtime specifics of “−Q 40, −G and −L flags”; and no postprocessing. MuSE was performed by using the following parameters: Docker at opengenomics/muse:latest; no preprocessing; runtime specifics of “MUSEv 1.0rc call”; and postprocessing using “MusEv 1.0rc sump”. Neusomatic was performed by using the following parameters: Docker at msahraeian/neusomatic:0.2.1; preprocessing using the provided preprocess.py; no runtime specifics; and postprocessing using the provided postprocess.py.

The overlap (e.g., concordance) between the predicted variant locations and the ground truth was calculated to provide values for false negatives, false positives, and true positives for variant calling. From these, the sensitivity and precision for each approach were calculated.

Results from initial training of VaRNN on one simulated (Bamsurgeon) sample of 22,884 loci, as described by Meng and Chen (PloS One, 2018), are shown in the top half of Table 5. These results shows that VaRNN outperforms the other variant callers on precision by a wide margin, with VaRNN achieving a precision of 0.94, followed by MuTect2 at 0.84. Similarly, VaRNN outperforms the other variant callers with an F1 score of 0.88, followed closely by Mutect2 at 0.85. Mutect2 performs marginally better in sensitivity, at a score of 0.86, followed closely by VaRNN at 0.83.

Results from training on real-world (MC3) tumor data are shown in the bottom half of Table 5. These results show that VaRNN outperforms the other variant callers on sensitivity and F1 by a wide margin, with VaRNN scoring 0.91 on both, followed by MuTect2 at 0.51 and 0.67, respectively. Mutect2 performs marginally better in precision, at a score of 0.98, followed closely by VaRNN at 0.92.

TABLE 5
Benchmarking of both training methods against other Bayesian variant
callers. Maximum values for sensitivity and precision are in bold.

| | MuSE | MuTect2 | SomaticSniper | VaRNN |
|---|---|---|---|---|
| BamSurgeon | | | | |
| TP | 17933 | 19591 | 13864 | 18969 |
| FP | 4983 | 3716 | 13189 | 1136 |
| FN | 4942 | 3284 | 9011 | 3906 |
| Sensitivity | 0.78 | 0.86 | 0.61 | 0.83 |
| Precision | 0.78 | 0.84 | 0.51 | 0.94 |
| F₁ | 0.78 | 0.85 | 0.56 | 0.88 |
| MC3 | | | | |
| TP | 194 | 291 | 102 | 519 |
| FP | 3 | 7 | 4 | 44 |
| FN | 378 | 281 | 470 | 53 |
| Sensitivity | 0.34 | 0.51 | 0.18 | 0.91 |

Finally, the variant callers were benchmarked for comparison purposes on entirely separate patient sample data, with 364 validated somatic mutations and 71 locations that were not classified as such by MC3. The outputs of the other somatic variant callers were filtered to only include these positions. VaRNN outperformed the other somatic variant callers in terms of sensitivity and F1 score (Table 6).

TABLE 6
True Positive (TP), True Negative (TN), False Negative
(FN), Sensitivity, Precision, and F1 Scores for
Recurrent Neural Network (RNN) variant callers
on a sample from the MC3 project: TCGA-D6-6827.

| | NeusomaticGT50¹ | Neusomatic² | MuTect2 | VaRNN |
|---|---|---|---|---|
| TP | 89 | 55 | 186 | 333 |
| FP | 8 | 5 | 12 | 26 |
| FN | 275 | 309 | 178 | 31 |
| Sensitivity | 0.245 | 0.151 | 0.511 | 0.915 |
| Precision | 0.918 | 0.917 | 0.939 | 0.928 |
| F₁ | 0.386 | 0.259 | 0.662 | 0.921 |

¹NeusomaticGT50: NeuSomatic_v0.1.4_standalone_SEQC-WGS-GT50-SpikeWGS10.pth
²Neusomatic: NeuSomatic_v0.1.4_standalone_SEQC-WGS-Spike.pth

VaRNN, a BiLSTM-based somatic variant caller developed using methods and systems of the present disclosure, outperforms both other Bayesian somatic variant callers as well as a CNN-based variant caller. The results show that VaRNN achieves high precision in calling somatic variants from simulated datasets, and high sensitivity in calling variants from difficult-to-detect real-world datasets. Additionally, VaRNN has a comparatively and consistently better F1 score for classifying non-trivial variants. The methods outlined here can be used in improving somatic variant detection for patients.

### Example 5

### Deep Bayesian Recurrent Neural Networks (RNNs) for Somatic Variant Calling in Cancer

Genomic profiles underlying individual tumors can be identified toward inform personalized cancer treatment strategies, e.g., in the field of precision oncology. This approach involves performing next generation sequencing (NGS) of a tumor sample and the subsequent identification of genomic aberrations, such as somatic mutations, and solving a classification-style problem to provide clinically useful information, such as potential candidates of targeted therapy. Some approaches of differentiating somatic (tumor) mutations from germline (heritable) variants and sequencing error can be performed using methods such as supervised machine learning methods, including deep-learning neural networks. In some cases, neural network-based methods may not be configured to provide an indication of confidence in the mutation call. As a result, an oncologist may potentially be targeting mutations having a low probability of being actually present in the patient (e.g., true positives). To address this unmet need, using methods and systems of the present disclosure, a deep Bayesian recurrent neural network (RNN) was developed for variant calling (e.g., in tumor samples), which shows no degradation in performance compared to other neural network-based methods. This approach enables greater flexibility through different priors to avoid overfitting to a single dataset. This approach can be incorporated into tumor assessment algorithms (e.g., used by oncologists, physicians, or other clinical health providers of a patient having cancer) to provide statistical confidence in precision oncology treatment choices.

Cancer is a group of diseases that result from specific changes in the genetic code of otherwise healthy cells. These changes can manifest in a variety of variants or mutations, such as DNA point mutations, insertion or deletion (e.g., indel) mutations, methylation aberrations, copy number aberrations, and structural variants. Next-generation DNA sequencing (NGS) can be commonly used in clinical practice to identify genetic variants, with oncologists using this information for, among other things, determining the best targeted and personalized treatment strategy for patients.

However, variants can be difficult to interpret due to challenges in performing deconvolution of or distinguishing somatic mutations from germline variants, and sequencing error. As a tumor can be the product of an accumulation of mutations over time, mutations can be found in anywhere from 0% to 100% of the sequencing reads in a tumor, and can be difficult to distinguish from germline variant frequencies and false-positive variants arising due to error rate from the sequencing process. Some approaches of differentiating somatic (tumor) mutations from germline (heritable) variants and sequencing error can be performed using methods such as supervised machine learning methods, including deep-learning neural networks. However, such neural network-based methods can be less than ideal because they may not be configured to provide an indication of confidence in the mutation call. As a result, an oncologist can potentially be targeting mutations having a low probability of being actually present in the patient (e.g., true positives). To address this unmet need, using methods and systems of the present disclosure, machine learning approaches were developed to classify somatic mutations from germline mutations or false-positive variants arising from sequencing error, which can achieve higher accuracy and performance as compared to other methods. This approach can be incorporated into tumor assessment algorithms (e.g., used by oncologists, physicians, or other clinical health providers of a patient having cancer) to provide statistical confidence in precision oncology treatment choices, including, such as by providing a more informative output, including determining a likelihood or probability that the classification (e.g., variant call) is correct.

Using methods and systems of the present disclosure, Bayesian Neural Networks (BNNs) are applied for somatic variant calling in cancer. This approach merges a Bayesian inference approach with the classification performance provided by neural networks. A goal of a BNN can be to learn a probability density function (pdf) over the space W of the weights. This approach involves learning beyond the first moment of a pdf (e.g., the mean) which can be learned by a non-Bayesian neural network. BNNs are utilized to provide oncologists with probability values corresponding to machine learning classification of mutation calls, to enable more informed use by oncologists in treatment selection, without impacting performance.

Variant calling was performed as follows. Next-generation DNA sequencing techniques produce overlapping short sequences of DNA called sequencing reads (“reads”). For a fixed position in a sequenced genome, a number of reads originating either from different cells or duplication of an original DNA strand can be determined as the depth. When, for a given patient, both tumor and normal tissue samples are sequenced for comparison, a number of true somatic variants can be expected to be observed (in the tumor tissue, but not in the normal tissue), although a significant portion or a majority of the variations observed can arise due to sequencing noise (e.g., false-positive variants). The variant calling approach comprises determining an indication of confidence in the differentiation of somatic variants from germline variants and false-positive variants arising from sequencing error.

The data was obtained as follows. The initial raw data was processed using a bioinformatics pipeline that identifies genomic loci where sequencing data from the tumor significantly differs from the normal. The data was extracted in the following form:

$$pair = [ \begin{matrix}{N}_{11} & … & {N}_{1 w} \\ ⋮ & ⋱ & ⋮ \\ {N}_{d 1} & … & {N}_{dw}\end{matrix} \vert \begin{matrix}{T}_{11} & … & {T}_{1 w} \\ ⋮ & ⋱ & ⋮ \\ {T}_{d 1} & … & {T}_{dw}\end{matrix} ]$$

d: the depth
w: the width or number of observed loci
Nᵢⱼ: The normal base at position i and depth d
Tᵢⱼ: The tumor base at position i and depth d

FIG. 18A shows a visual representation of the pair matrix with w=10 and d=10.

The labels were obtained as follows. For Training, the Multi-Center Mutation Calling in Multiple Cancers (MC3) dataset was used. The MC3 dataset was generated by next generation sequencing (NGS) of tumor/normal sample pairs. Further, a validation pipeline was used, including orthogonal methods of sequencing the DNA, such as Sanger sequencing. This approach yields an established dataset in which the identified variants are deemed to be very high confidence.

The labels for this dataset are binary: “1” if the variant is validated as positive, and “0” if the variant is validated as negative. Non-validated data was not included in the study.

The models were developed as follows. The input data comprises the aforementioned pair matrices with a specific size of (100, 20) with three color channels. The input data was reshaped from (100, 20, 3) to (100, 60), diluting the color information to analyze using LSTMs. Secondly, the dataset was balanced by undersampling the majority class. This resulted in a total of 103,868 (image, label) pairs. For these data, a 0.8 training-test split was used (e.g., 80% of the dataset was randomly selected as the training dataset, and the remaining 20% of the dataset was selected as the test dataset).

Two models were implemented: a first model having two layers of bidirectional LSTMs (BiLSTMs), and a second similar model with variational dense layers instead of standard layers. The DenseFlipout layers provided by the Tensorflow Probability library (tfp) (github.com/tensorflow/probability) were used. The Adam optimizer was used to optimize the cost function of each network.

Because variational layers were used, during training with data D, an approximate probability density function posterior p(\vec{w}| [symbol] ) for the weights of the network was learned by minimizing the cost function ELBO (Evidence Lower Bound)

ELBO=−E_{W}_{_{\vec{w}}}_{(\vec{θ})}log(P( [symbol] |\vec{w}))+KL(Q_{\vec{w}}(\vec{θ})∥p(\vec{w})) (1)

where P( [symbol] |\vec{w}) and p(\vec{w}) are the likelihood of the data and the prior, respectively, for the weights \vec{w}, and Q_{\vec{w}}(\vec{θ}) is an approximate pdf to the true posterior. Maximizing the ELBO can then be regarded as the same as minimizing the KL divergence between the true posterior and the approximate posterior for the weights. A multivariate gaussian was assumed for the variational posterior Q_{\vec{w}}(\vec{θ}). Practically, as the integrals in Equation (1) are in general analytically intractable, they were computed via Monte Carlo (MC) sampling from the approximate pdf Q_{\vec{w}}(\vec{θ}). This can be automatically done by the variational layers in tfp2. Further, as it can be computationally costly (e.g., with high computational complexity) to calculate the ELBO for large data sets such as those in DNA sequencing, tfp allows the performance of automatic minibatch optimization. Therefore, Equation (1) can be calculated as an average over many ELBOs calculated on batches of data [symbol] ₖ with sampled weights \vec{w}ᵢ.

$${EL}^{\hat{}} BO = \frac{1}{{N}_{MC , elbo}} {\sum }_{i = 1}^{{N}_{MC , elbo}} [ - ( log ( p ( {\vec{ω}}_{i} ) ) + log ( p ( {𝒟}_{k} ) \vert {\vec{ω}}_{i} ) ) ) + log ( q ( {\vec{ω}}_{i} ) \vert \vec{θ} ) ] \qquad ( 2 )$$

This approximation can be an unbiased stochastic estimator for the ELBO. As a result, the calculated gradient may not be miscalculated, thereby allowing convergence to the correct solution.

The usefulness of the Flipout estimator in the layers can be that the unbiased estimation of the ELBO has lower variance with respect to other methods. The variance of the ELBO, and not its actual value, can dictate the convergence of the optimization process during training. After learning the pdf Q_{\vec{w}}(\vec{θ})˜p(\vec{w}| [symbol] ) for the weights, via ELBO optimization, a new prediction y* can be generated with a new X* datum, by calculating

p(y*)=∫d\vec{w}p(y*|X*,\vec{w})p(\vec{w}| [symbol] ) (3)

This can be done in practice by Monte Carlo sampling, where the integral can be substituted with an averaged sum over weights sampled from the approximate posterior, to make a prediction from a network in an ensemble of networks, and then taking the average

$$\dot{p} ( {y}^{*} ) = \frac{1}{{N}_{MC}} {\sum }_{{\vec{ω} }_{k} ∼ {Q}_{\vec{ω}} ( \vec{θ} )}^{ } p ( {y}^{*} \vert {X}^{*} , {\vec{ω}}_{k} ) \qquad ( 4 )$$

In a classification problem, this means that for each possible outcome y* ∈ [symbol] , where [symbol] is the set of all outcomes, this estimate \hat{p}(y*) can be calculated, ensuring that the probabilities are properly normalized such that [symbol] \hat{p}(y*)=1.

To check performance degradation, the non-Bayesian neural networks and Bayesian neural networks were trained without optimization of a set of hyper-parameters. After training for 20 epochs, a training sparse categorical accuracy of 0.76 and 0.78 was achieved for the non-Bayesian NN and BNN, respectively, with a 0.75 accuracy on the test set for both. The non-Bayesian model does not provide confidence in any given result, while the BNN provides a measure of uncertainty. Utilizing the probabilities for each BNN outcome y* given the input datum X*, a “confidence” measure (e.g., likelihood or probability) of the prediction (e.g., the called variant) is generated. In some cases, an uninformative prior can be found, such as when only one MC call is performed. To call more than once, approaches such as those disclosed in github.com/tensorflow/probability/issues/213#issuecomment-440395740 uniform can be used, with constant likelihood, therefore an ‘uninformative’ posterior. In that case, 1/| [symbol] | can be outputted as the probability for each outcome. In this case, the scatter between probabilities can be zero, and this corresponds to maximum uncertainty. In cases with a high confidence output, the scatter can be much higher. Therefore, a simple measure can be the variation of information entropy with respect to the uniform case. As shown in FIG. 18B, the confidence can be degraded when a black mask has been artificially added on the input images, thereby making the input distribution different than those seen during training. The BNN becomes uncertain for out-of-distribution samples. FIG. 18B shows an example of results produced by the Bayesian LSTM-based approach applied to original data (left) and masked data (right).

The model was tested on both in-distribution and out-of-distribution samples. The results are summarized in FIG. 18C, showing plots of the frequency of the probabilities that the network outputs for each sample. For the first case, a clearly skewed distribution can be observed, with a dip in the middle. This means that the BNN is somewhat certain about its predictions. In contrast, for the second case (right), output probabilities are observed mostly around 0.5, indicating that the model is generating uncertain variant calls. FIG. 18C shows histograms of the output probabilities from the BNN tested on in-distribution samples (left) and masked data that are out-of-distribution (right).

The results show that BNNs do not degrade the performance of neural networks in this application, while enabling the determination of useful probabilities corresponding to the output predictions. Further, the results show that BNNs enable detection of distribution samples without additional work. Although BNNs can be computationally expensive to use as compared to non-Bayesian neural networks, BNNs are more flexible and allow the choice of different priors or likelihoods to suit the input data and to give naturally regularizing terms (e.g., the KL term in Equation (1)) to avoid overfitting.

For the applications described herein, the use of BNNs instead of non-Bayesian neural networks does not degrade the performance of predictions, while enabling the determination of useful probabilities corresponding to the output predictions. In particular, in regions where data is sparse, or in out-of-distribution regions, the network becomes uncertain, and this can be reflected in the probability values. This feature may not be available via non-Bayesian neural network-based approaches, in which the neural network outputs values with one hundred percent confidence, regardless of out-of-distribution regions.

Further, the confidence of the network can be assessed a standard way that depends on the number of classes. For example, a threshold of 0.95 can be used for high confidence. Subsequently, the BNN can be trained on the entire data set, and optimization can be performed. Optimization of the neural network can be performed with high accuracy, such as via optimization through a cyclic learning rate, achieving an initial (binary crossentropy) accuracy of 0.9. Further, to exploit the input data format, convolutional LSTMs, where the internal input LSTM operations are given by convolutions, can be used.

In summary, the BNN-based methods and systems provided herein demonstrate a highly accurate deep Bayesian neural network-based approach for improved genetic variant identification and assessment, including determinations of a likelihood or probability for each output variant called. The methods provided herein are a useful approach in providing confidence metrics on somatic variant calls.

### Example 6

### SomaticNET: A Machine Learning Approach for Highly Accurate Evaluation of Somatic Mutations in Cancer

Methods of performing DNA sequencing to identify genetic variants can become increasingly valuable in clinical applications, including matching patients to targeted therapies, immunotherapies, and/or clinical trials. However, accurate calling of genetic variants from DNA sequencing data can face challenges. For example, false-positive variants can be identified or “called”, and without corroboration across different variant calling algorithms, patients who receive disease or treatment assessments based on such variant calling can be at risk of being treated with therapies that are unsuitable for their disease, disorder, or abnormal condition (e.g., cancer).

Using systems and methods of the present disclosure, a machine learning-learned based method, called SomaticNET, was developed for the accurate identification of somatic variants in cancer patient tumor samples. The SomaticNET approach comprises applying a neural network architecture to encoded raw sequencing read information from tumor/normal sample pairs, enabling multi-class classification of variant sequencing reads into those which are germline, somatic, or false positives arising from sequencing error.

The model was trained and tested on in silico spike-in data, as well as multi-cancer and multi-center data. Results were then benchmarked against other variant callers. Results showed that the SomaticNET approach outperforms other variant callers, achieving AUROCs of about 1.00 and 0.99, respectively. The methods provided herein can provide for accurate somatic variant calling and can ensure patients are given the right therapy at the right time to treat their cancer.

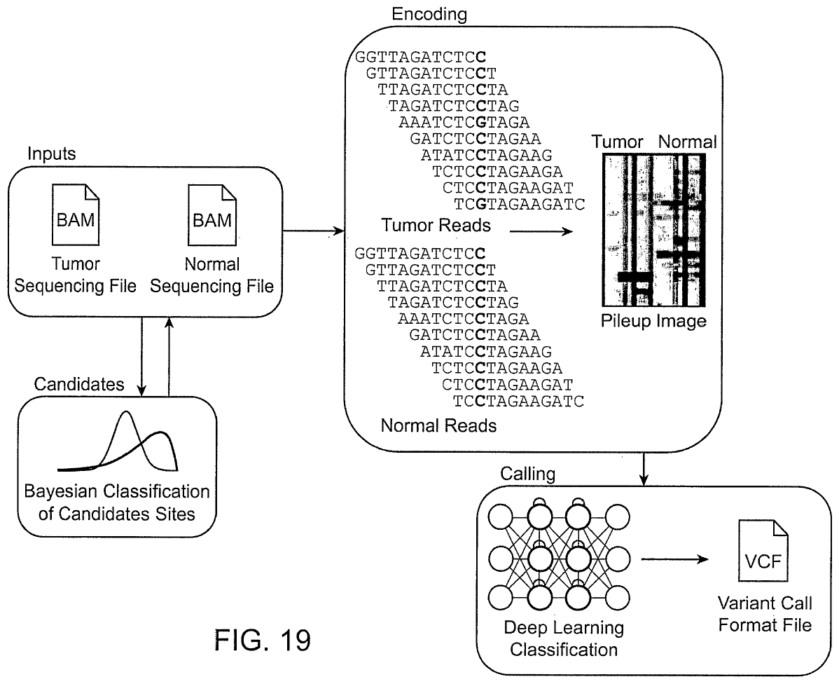

FIG. 19 shows an example workflow of the machine learning-based variant calling, including obtaining tumor and normal sequencing files as inputs, encoding the tumor reads and normal reads into a pileup image, and performing variant calling by applying deep learning classification to the pileup image to generate a variant call format (VCF) file. A Bayesian classifier can be configured to identify both germline variants (present in both tumor and normal samples) and candidate somatic variants (present in the tumor but not the normal) which appear at a higher minor allele frequency (MAF) than false-positive variants arising from sequencing error. This can be used to subset the sequencing files to candidate sites only. The sequencing reads at these candidate sites are then encoded into a side-by-side pileup for processing by the pre-trained neural network. The output can be returned in a standardized VCF file for downstream analysis.

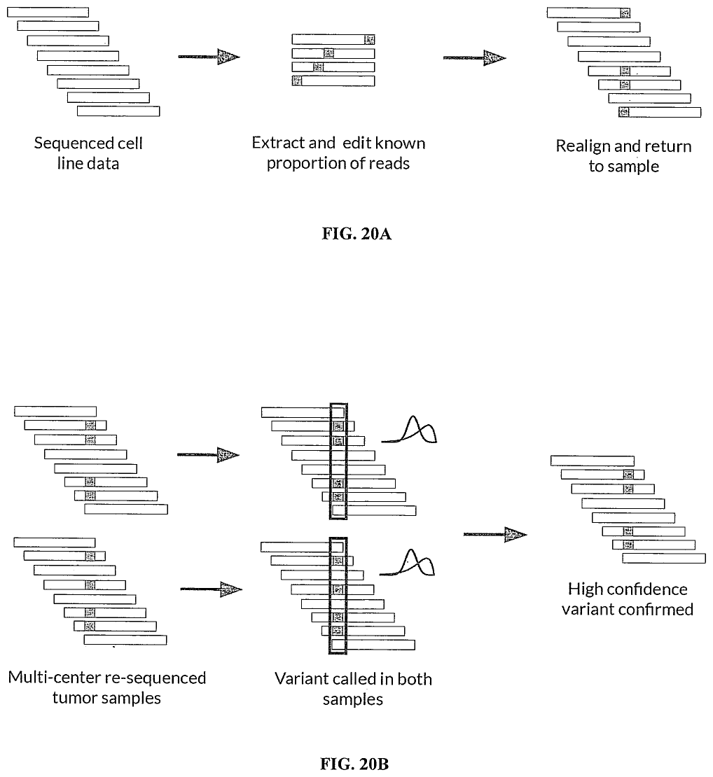

The training data was generated as follows. The neural network was trained and tested (with an 80/20 split, such that 80% of the dataset was randomly selected as the training dataset, and the remaining 20% of the dataset was selected as the test dataset) on both simulated and real-world datasets. FIG. 20A shows an example of how simulated variants were spiked in silico into the NA12878 cell line dataset, which included sequencing cell line data to produce a plurality of sequencing reads, extracting and editing a known subset of the plurality of sequencing reads, and realigning the edited sequencing reads and returning them to the plurality of sequencing reads. Further, FIG. 20B shows an example of how high-confidence variants were identified from the MC3 (multi-center mutation calling in multiple cancers) dataset across technical replicates, which included re-sequencing multi-center tumor samples, performing variant calling in multiple sets of samples, and confirming the high-confidence variants.

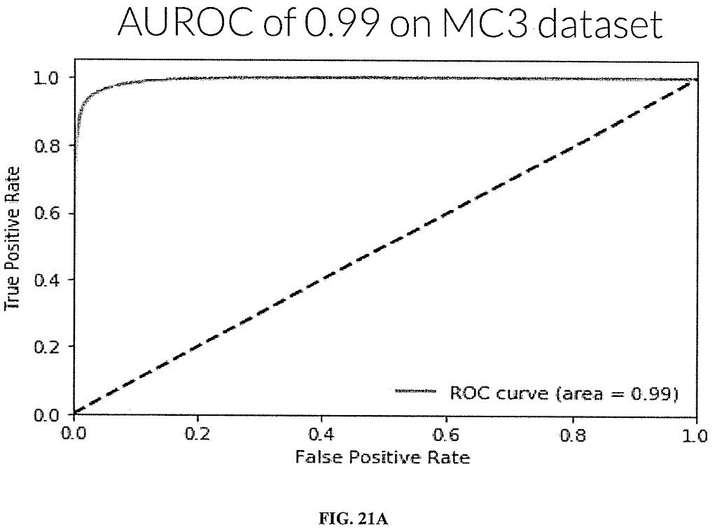

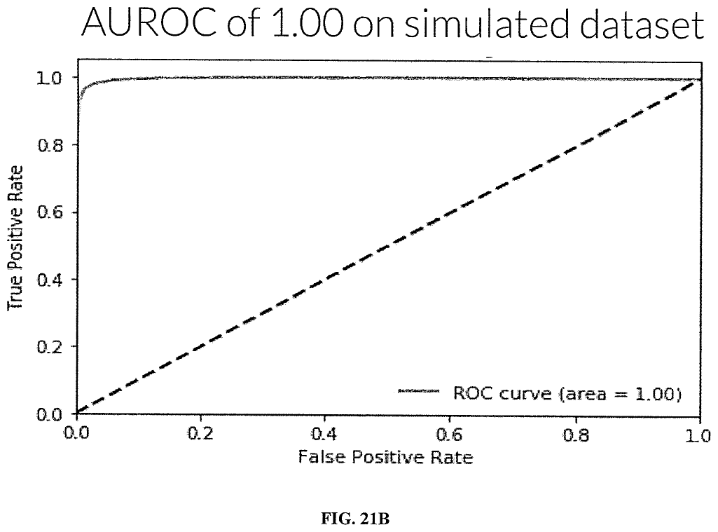

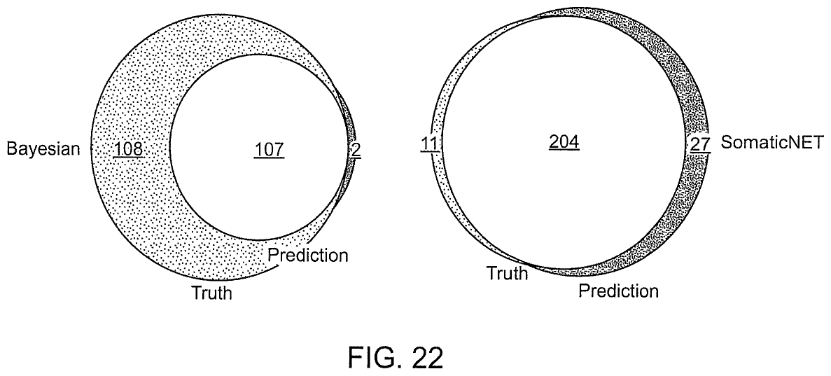

FIGS. 21A-21B show results demonstrating that the neural network achieved a very high AUROC on both datasets. For example, an AUROC of 0.99 was achieved on the MC3 dataset (FIG. 21A), while an AUROC of 1.00 was achieved on the simulated dataset (FIG. 21B). In addition, FIG. 22 shows that SomaticNET demonstrated superior performance in catching “edge cases” (e.g., borderline cases such as somatic variants that are difficult to distinguish from germline variants or false-positive variants arising from sequencing error) as compared to Bayesian variant calling alone. Further, benchmarking against four examples of other variant callers shows greater precision using the SomaticNET approach, as shown in Table 7.

TABLE 7
Performance Benchmark Comparison of SomaticNET
against four other variant callers

| | VarScan2 | DeepVariant | Lancet | Strelka2 | SomaticNET |
|---|---|---|---|---|---|
| True Positives | 11023 | 13454 | 16477 | 16446 | 18969 |
| False Positives | 1105 | 2347 | 3521 | 3200 | 1136 |
| Precision | 90.89% | 85.15% | 82.39% | 83.71% | 94.35% |

Increased accuracy in variant calling can play a role in matching patients to therapies and trials. SomaticNET uses a machine learning-based method for more accurate identification of somatic variants in tumor samples, as shown by a comparison to results obtained using other somatic variant callers.

### Example 7

### Somatic Variant Caller Based on Recurrent Neural Networks

Using systems and methods of the present disclosure, a somatic variant caller based on recurrent neural networks (RNNs) can be developed, which is able to call variants faster and more accurately as compared to other methods. In additional to determining whether a given variant is a somatic mutation (e.g., as a binary or Boolean value), the RNN-based somatic variant caller provides for each for variant a likelihood (e.g., a probability) of being a somatic mutation. Further, the RNN-based somatic variant caller comprises a model that has the ability to be retrained on specific datasets. This model can be obtained by testing on datasets for which ground truth outcomes are available (synthetic or MC3).

In some cases, developing the RNN-based somatic variant caller comprises formatting the input SAM or BAM files in a matricial manner (e.g., by generating pileup images from the DNA sequence read data), where the tumor sample data is arranged adjacent to the normal sample data.

In some cases, developing the RNN-based somatic variant caller comprises using a multilayer bi-directional long short-term memory (BiLSTM) network on the previously generated matrices, where the depth is the number of time steps and the window with the input dimensionality.

In some cases, developing the RNN-based somatic variant caller comprises producing Python scripts including the use of a BiLSTM for variant calling and/or the use of a Bayesian neural network for variant calling.

### Example 8

### Parallelized Processing of DNA Sequence Data

Performing variant calling on large input files corresponding to DNA sequence data can involve significant computational complexity and therefore take a long time to complete. For example, performing variant calling using Mutect on a whole-genome sequencing (WGS) SAM file with a depth of about 30× can take about 24 hours to call on a single instance of 4 cores. Given that variant calling can involve significant time and computational resources to complete and can be a recurrent process as part of research and development activities, the parallelization of the variant calling process can significantly increase iteration frequency. Using systems and methods of the present disclosure, input files corresponding to DNA sequence data are processed in a highly parallelized manner for increased throughput and faster algorithms. This can comprise splitting the input files (e.g., sequence alignment map (SAM), BAM, and/or CRAM) into small regions to parallelize redundant bioinformatic processing. Such approaches can effectively reduce or minimize the size of the induced microbams and maximizing compatibility with variant calling algorithms. For example, the results can be obtained using a single instance with 96 cores, or a Pachyderm cluster.

FIG. 23A shows an example of splitting an original SAM file into N different microSAM or microBAM files. The parallelized process can comprise computing the coverage of the input SAM (e.g., using a list of all regions). Next, the parallelized process can comprise defining a maximum interval. Next, the parallelized process can comprise putting in the same set all regions that are closer to each other than the previously defined maximum interval. This operation generates a list of region sets (as shown in FIG. 23B). Next, the parallelized process can comprise splitting or slicing the original SAM into a plurality of different sets, corresponding to the list of region sets. For each of the different sets, a microSAM or microBAM file can be created. Next, the parallelized process can comprise applying computation (e.g., non-parallelized computation or parallelized computation) on the microSAMs, which can include overwriting read groups, indexing the SAM or BAM or microSAM or microBAM files, and generating pileups from the microSAM or microBAM files.

As an example, using the maximum 8 cores provided by Mutect can lead to about 24 hours of computation time to perform variant calling on a given input file. The parallelized process may not be limited by the number of cores available, but by the size of the largest set of regions. For the same SAM or BAM file, the computation time can be reduced significantly to about 30 minutes (e.g., resulting in a speedup of about 48×). Therefore, the parallelization process can produce a speedup of at least about 1.1×, at least about 1.2×, at least about 1.3×, at least about 1.4×, at least about 1.5×, at least about 1.6×, at least about 1.7×, at least about 1.8×, at least about 1.9×, at least about 2×, at least about 3×, at least about 4×, at least about 5×, at least about 10×, at least about 15×, at least about 20×, at least about 25×, at least about 30×, at least about 35×, at least about 40×, at least about 45×, at least about 50×, at least about 60×, at least about 70×, at least about 80×, at least about 90×, at least about 100×, or more than about 100×, as compared to other non-parallelized methods of variant calling.

The parallelized process can be performed with a runtime of no more than about 24 hours, no more than about 22 hours, no more than about 20 hours, no more than about 18 hours, no more than about 16 hours, no more than about 14 hours, no more than about 12 hours, no more than about 10 hours, no more than about 9 hours, no more than about 8 hours, no more than about 7 hours, no more than about 6 hours, no more than about 5 hours, no more than about 4 hours, no more than about 3 hours, no more than about 2 hours, no more than about 60 minutes, no more than about 45 minutes, no more than about 30 minutes, no more than about 20 minutes, no more than about 15 minutes, no more than about 10 minutes, or no more than about 5 minutes. As an example, the parallelized process can be performed with a runtime of no more than 30 minutes, including splitting the SAM file (about 6 minutes), creating the micro-SAM files (about 3 minutes), performing the algorithms (about 10 minutes), and saving the resulting datasets (about 5 minutes).

In some cases, the methods and systems of the present disclosure may be performed using a single-core or multi-core machine, such as a dual-core, 3-core, 4-core, 5-core, 6-core, 7-core, 8-core, 9-core, 10-core, 12-core, 14-core, 16-core, 18-core, 20-core, 22-core, 24-core, 26-core, 28-core, 30-core, 32-core, 34-core, 36-core, 38-core, 40-core, 42-core, 44-core, 46-core, 48-core, 50-core, 52-core, 54-core, 56-core, 58-core, 60-core, 62-core, 64-core machine, 96-core, 128-core, 256-core, 512-core, 1,024-core, or a multi-core machine having more than 1,024 cores. In some cases, the methods and systems of the present disclosure may be performed using a distributed network, such as a cloud computing network, which is configured to provide a similar functionality as a single-core or multi-core machine.

In some cases, the methods and systems of the present disclosure may be performed using an input SAM, BAM, or CRAM file having a size of about 1 Gb, 2 Gb, 3 Gb, 4 Gb, 5 Gb, 6 Gb, 7 Gb, 8 Gb, 9 Gb, 10 Gb, 15 Gb, 20 Gb, 25 Gb, 30 Gb, 35 Gb, 40 Gb, 45 Gb, 50 Gb, 55 Gb, 60 Gb, 65 Gb, 70 Gb, 75 Gb, 80 Gb, 85 Gb, 90 Gb, 95 Gb, 100 Gb, 110 Gb, 120 Gb, 130 Gb, 140 Gb, 150 Gb, 160 Gb, 170 Gb, 180 Gb, 190 Gb, 200 Gb, 250 Gb, 300 Gb, 350 Gb, 400 Gb, 450 Gb, 500 Gb, 550 Gb, 600 Gb, 650 Gb, 700 Gb, 750 Gb, 800 Gb, 850 Gb, 900 Gb, 950 Gb, 1000 Gb, or more than about 1000 Gb. Generally, the runtime may increase linearly with the file size of the dataset that is processed for variant calling. In some cases, the runtime may increase logarithmically with the file size of the dataset that is processed for variant calling

In some cases, the methods and systems of the present disclosure may be performed using a depth of sequencing of about 1×, 2×, 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, 110×, 120×, 130×, 140×, 150×, 160×, 170×, 180×, 190×, 200×, 250×, 300×, 350×, 400×, 450×, 500×, 550×, 600×, 650×, 700×, 750×, 800×, 850×, 900×, 950×, 1000×, or more than about 1000×. Generally, the runtime may increase linearly with the depth of sequencing of the dataset that is processed for variant calling. In some cases, the runtime may increase logarithmically with the depth of sequencing of the dataset that is processed for variant calling.

In some cases, the methods and systems of the present disclosure may be performed using a total number of sequencing reads of about 100, 200, 300, 400, 500, 600, 700, 800, 900, 1 thousand, 2 thousand, 3 thousand, 4 thousand, 5 thousand, 6 thousand, 7 thousand, 8 thousand, 9 thousand, 10 thousand, 15 thousand, 20 thousand, 25 thousand, 30 thousand, 35 thousand, 40 thousand, 45 thousand, 50 thousand, 55 thousand, 60 thousand, 65 thousand, 70 thousand, 75 thousand, 80 thousand, 85 thousand, 90 thousand, 95 thousand, 100 thousand, 110 thousand, 120 thousand, 130 thousand, 140 thousand, 150 thousand, 160 thousand, 170 thousand, 180 thousand, 190 thousand, 200 thousand, 250 thousand, 300 thousand, 350 thousand, 400 thousand, 450 thousand, 500 thousand, 550 thousand, 600 thousand, 650 thousand, 700 thousand, 750 thousand, 800 thousand, 850 thousand, 900 thousand, 950 thousand, 1 million, 2 million, 3 million, 4 million, 5 million, 6 million, 7 million, 8 million, 9 million, 10 million, 15 million, 20 million, 25 million, 30 million, 35 million, 40 million, 45 million, 50 million, 55 million, 60 million, 65 million, 70 million, 75 million, 80 million, 85 million, 90 million, 95 million, 100 million, 110 million, 120 million, 130 million, 140 million, 150 million, 160 million, 170 million, 180 million, 190 million, 200 million, 250 million, 300 million, 350 million, 400 million, 450 million, 500 million, 550 million, 600 million, 650 million, 700 million, 750 million, 800 million, 850 million, 900 million, 950 million, 1 billion, 2 billion, 3 billion, 4 billion, 5 billion, 6 billion, 7 billion, 8 billion, 9 billion, 10 billion, 15 billion, 20 billion, 25 billion, 30 billion, 35 billion, 40 billion, 45 billion, 50 billion, 55 billion, 60 billion, 65 billion, 70 billion, 75 billion, 80 billion, 85 billion, 90 billion, 95 billion, 100 billion, 110 billion, 120 billion, 130 billion, 140 billion, 150 billion, 160 billion, 170 billion, 180 billion, 190 billion, 200 billion, 250 billion, 300 billion, 350 billion, 400 billion, 450 billion, 500 billion, 550 billion, 600 billion, 650 billion, 700 billion, 750 billion, 800 billion, 850 billion, 900 billion, 950 billion, or 1 trillion sequencing reads. Generally, the runtime may increase linearly with the number of total number of sequencing reads of the dataset that is processed for variant calling. In some cases, the runtime may increase logarithmically with the number of total number of sequencing reads of the dataset that is processed for variant calling.

Generally, for a given sequencing depth and a given computational power, the runtime may increase linearly with the number of sequencing reads that are processed. For example, on an 8-core machine, a 60 Gb SAM file of 150× depth with 926,000,000 sequencing reads is processed by Mutect with a total runtime of about 35 hours. In comparison, using the parallelization process with methods and systems of the present disclosure, the same SAM file was processed with a total runtime of about 5 hours. Therefore, using methods and systems of the present disclosure, the parallelization process produced a speedup of about 7× was achieved as compared to performing the same process using Mutect.

As another example, on an 8-core machine, a 60 Gb SAM file of 150× depth with 926,000,000 sequencing reads is processed using the parallelization process with methods and systems of the present disclosure, with a total runtime of no more than about 30 hours, no more than about 28 hours, no more than about 26 hours, no more than about 24 hours, no more than about 22 hours, no more than about 20 hours, no more than about 18 hours, no more than about 16 hours, no more than about 14 hours, no more than about 12 hours, no more than about 10 hours, no more than about 8 hours, no more than about 7 hours, no more than about 6 hours, no more than about 5 hours, no more than about 4 hours, no more than about 3 hours, no more than about 2 hours, no more than about 1 hour, no more than about 45 minutes, no more than about 30 minutes, no more than about 20 minutes, no more than about 15 minutes, no more than about 10 minutes, or no more than about 5 minutes.

As another example, on an 8-core machine, a 60 Gb SAM file of 150× depth with 926,000,000 sequencing reads is processed using the parallelization process with methods and systems of the present disclosure, with a total runtime of about 30 hours, about 28 hours, about 26 hours, about 24 hours, about 22 hours, about 20 hours, about 18 hours, about 16 hours, about 14 hours, about 12 hours, about 10 hours, about 8 hours, about 7 hours, about 6 hours, about 5 hours, about 4 hours, about 3 hours, about 2 hours, about 1 hour, about 45 minutes, about 30 minutes, about 20 minutes, about 15 minutes, about 10 minutes, or about 5 minutes.

As another example, on an 8-core machine, a 60 Gb SAM file of 150× depth with 926,000,000 sequencing reads is processed using the parallelization process with methods and systems of the present disclosure, with a speedup of about 5×, 6×, 7×, 8×, 9×, 10×, 12×, 14×, 16×, 18×, 20×, 25×, 30×, 35×, 40×, 45×, 50×, 60×, 70×, 80×, 90×, or 100× as compared to performing the same process using Mutect.

### Example 9

### Generating Pileup Images From SAM or BAM Files for Both Machine Learning Models and Humans

In some cases, pileup images generated from DNA sequencing read data can be difficult to represent in such a manner as to be efficiently interpreted by machine learning algorithms (e.g., models) and/or humans. For example, FIGS. 24A-24E show examples of pileup images that can be challenging for humans to visually interpret. In order to generate a compact, readable, yet complete representation of genetic variants, such as somatic variants, a set of procedures or rules can be performed to process such pileup images (e.g., which can include complex mutations) to make them both more compact in size and more readable and visually interpretable. Such approaches can have advantages, including faster generation and training, enhanced performance of many deep learning models (e.g., classifiers), and easier and faster debugging of machine learning models. For example, the models can be trained earlier as a result of faster data generation. Further, for a given amount of computation time used to perform the data generation, the faster data generation enables more data to be produced, thereby improving model performance. In some cases, this may result in a logarithmic gain on performance, depending on the model used. In some cases, this may result in a linear gain on performance, depending on the model used.

First, the data can be loaded, including a tumor BAM file (e.g., generated by sequencing a tumor tissue sample from a subject), a normal BAM file (e.g., generated by sequencing a normal tissue sample from a subject), and a list of candidates (e.g., genomic positions where there may be somatic variants (such as single nucleotide polymorphisms (SNPs), germline mutations, sequencing noise, or PCR noise).

Next, a tumor image can be created by obtaining a set of pileup sequencing reads from the tumor BAM file, applying a Concise Idiosyncratic Gapped Alignment Report (CIGAR) correction, applying a coloration algorithm, applying a quality filter, and adding a reference. In some cases, obtaining the set of pileup sequencing reads comprises, for a given location use of ‘pysam’, querying the ordered reads that share the location. In some cases, applying the CIGAR correction comprises: in the case of 0 (e.g., a match), keeping the base call as is; in the case of 1 (e.g., an insertion or INS), removing all inserted bases and replacing them by one base of a specific color for insertions; and in the case of 2 (e.g., a deletion or DEL), applying a right offset of the size of the deletion. In some cases, applying the coloration algorithm comprises assigning each base to a given color found by categorically combining the three RGB color channels (red, green, and blue). In some cases, applying the quality filter comprises, for each base, multiplying the color of the base by the quality score (e.g., between 0 and 1), thereby applying a desaturation filter. In some cases, adding the reference comprises following the same coloration process, but with a constant quality of 1, to create and add a reference row to the top.

Next, a normal image can be created by obtaining a set of pileup sequencing reads from the normal BAM file, and processing the pileup sequencing reads from the normal BAM file in a similar manner as for the pileup sequencing reads from the tumor BAM file.

Finally, the tumor and normal images are concatenated. This can be performed by concatenating on the second axis (horizontal) both the tumor and normal matrices, thereby generating a pileup matrix.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method for determining a somatic nucleotide variant in nucleic acids from a cell-free sample of a subject, comprising:
(a) obtaining a first plurality of sequencing reads of the nucleic acids from the cell-free sample of the subject and a second plurality of sequencing reads of nucleic acids from a normal tissue of the subject;
(b) generating a data input from the first plurality of sequencing reads and the second plurality of sequencing reads; and
(c) determining the somatic nucleotide variant in the nucleic acids from the cell-free sample by applying a trained neural network to the data input.

2. The method of claim 1, wherein the data input comprises a first tensor and a second tensor, wherein each of the first plurality of sequencing reads is represented in a different row of the first tensor, and wherein each of the second plurality of sequencing reads of the nucleic acids is represented in a different row of the second tensor.

3. The method of claim 2, wherein the first and second tensors comprise digital representations of images, and wherein (b) comprises creating a pileup image from the first plurality of sequencing reads, creating a normal pileup image from the second plurality of sequencing reads, applying a coloration algorithm to the pileup image and the normal pileup image, and concatenating the pileup image and the normal pileup image, thereby generating a pileup matrix.

4. The method of claim 2, wherein the trained neural network comprises a Siamese neural network comprising two identical trained sister neural networks, wherein each of the two identical trained sister neural networks generates an output, and wherein the Siamese neural network is configured to apply a function to the outputs of the two identical trained sister neural networks to determine a classification indicative of whether the outputs are identical or different.

5. The method of claim 4, wherein the Siamese neural network is configured to determine the classification by applying the first of the two identical trained sister neural networks to the first tensor to generate a first output, applying the second of the two identical trained sister neural networks to the second tensor to generate a second output, and comparing a distance between the first and second outputs against a threshold, wherein the threshold is pre-set in the Siamese neural network or is optimized during training of the Siamese neural network.

6. The method of claim 1, wherein the trained neural network comprises a convolutional neural network (CNN), a recurrent neural network (RNN), a long short-term memory (LSTM) network, or any combination thereof.

7. The method of claim 6, wherein the trained neural network comprises a long short-term memory (LSTM) recurrent neural network (RNN).

8. The method of claim 7, wherein the LSTM RNN comprises a bidirectional LSTM (BiLSTM) RNN.

9. The method of claim 1, further comprising generating a likelihood value corresponding to the determined somatic nucleotide variant in the nucleic acids from the cell-free sample, wherein the likelihood value is a probability value of the determined somatic nucleotide variant being present in the cell-free sample of the subject, and wherein generating the likelihood value comprises learning a probability density function over a set of weights of the trained neural network.

10. The method of claim 9, wherein learning the probability density function comprises applying Bayesian inference to the set of weights.

11. The method of claim 10, wherein the trained neural network comprises a BiLSTM RNN comprising two layers of BiLSTM cells.

12. The method of claim 1, wherein (b) comprises processing a sequence alignment map (SAM) or binary alignment map (BAM) file of the first plurality of sequencing reads or a SAM or BAM file of the second plurality of sequencing reads by: splitting the SAM or BAM file into a plurality of distinct microSAM or microBAM files, wherein each distinct microSAM or microBAM file comprises a different set from among a plurality of sets of the sequencing reads, and performing parallel processing of the plurality of distinct microSAM or microBAM files.

13. A method for determining a somatic nucleotide variant in nucleic acids from a sample of a subject, comprising:
(a) obtaining a plurality of sequencing reads of the nucleic acids from the sample of the subject;
(b) generating a data input from the plurality of sequencing reads; and
(c) determining the somatic nucleotide variant in the nucleic acids from the cell-free sample by applying a trained neural network to the data input, wherein the trained neural network comprises a long short-term memory (LSTM) network, a recurrent neural network (RNN), or a combination thereof.

14. The method of claim 13, wherein the data input comprises a tensor, wherein the tensor comprises a representation of the plurality of sequencing reads, and wherein each of the plurality of sequencing reads is represented in a different row of the tensor.

15. The method of claim 13, wherein the sample is a cell-free sample.

16. The method of claim 13, wherein the trained neural network comprises a long short-term memory (LSTM) recurrent neural network (RNN).

17. The method of claim 16, wherein the LSTM RNN comprises a bidirectional LSTM (BiLSTM) RNN.

18. The method of claim 17, wherein the BiLSTM RNN comprises two layers of BiLSTM cells.

19. The method of claim 11, further comprising generating a likelihood value corresponding to the determined somatic nucleotide variant in the nucleic acids from the cell-free sample, wherein the likelihood value is a probability value of the determined somatic nucleotide variant being present in the cell-free sample of the subject, and wherein generating the likelihood value comprises learning a probability density function over a set of weights of the trained neural network.

20. The method of claim 19, wherein learning the probability density function comprises applying Bayesian inference to the set of weights.

21. The method of claim 13, wherein (b) comprises processing a sequence alignment map (SAM) or binary alignment map (BAM) file of the plurality of sequencing reads by: splitting the SAM or BAM file into a plurality of distinct microSAM or microBAM files, wherein each distinct microSAM or microBAM file comprises a different set from among a plurality of sets of the sequencing reads, and performing parallel processing of the plurality of distinct microSAM or microBAM files.

22. A method for determining a somatic nucleotide variant in cell-free nucleic acids from a subject, comprising:
(a) obtaining a plurality of sequencing reads of the cell-free nucleic acids from the subject;
(b) generating a data input comprising one or more tensors, wherein each of the plurality of sequencing reads of the cell-free nucleic acids is represented in a different row of the one or more tensors; and
(c) determining the somatic nucleotide variant in the cell-free nucleic acids by applying a trained neural network to the data input.

23. The method of claim 22, wherein the trained neural network comprises one or more of: a deep neural network (DNN), a convolutional neural network (CNN), a feed forward network, a cascade neural network, a radial basis network, a deep feed forward network, a recurrent neural network (RNN), a long short-term memory (LSTM) network, a gated recurrent unit, an auto encoder, a variational auto encoder, a denoising auto encoder, a sparse auto encoder, a Markov chain, a Hopfiled network, a Boltzmann Machine, a restricted Boltzmann Machine, a deep belief network, a deconvolutional network, a deep convolutional inverse graphics network, a generative adversarial network, a liquid state machine, an extreme learning machine, an echo state network, a deep residual network, a Kohonen network, a support vector machine, a neural Turing machine, and any combination thereof.

24. The method of claim 23, wherein the trained neural network comprises a convolutional neural network (CNN), a recurrent neural network (RNN), a long short-term memory (LSTM) network, or any combination thereof.

25. The method of claim 24, wherein the trained neural network comprises a long short-term memory (LSTM) recurrent neural network (RNN).

26. The method of claim 25, wherein the LSTM RNN comprises a bidirectional LSTM (BiLSTM) RNN.

27. The method of claim 22, further comprising generating a likelihood value corresponding to the determined somatic nucleotide variant in the nucleic acids from the cell-free sample, wherein the likelihood value is a probability value of the determined somatic nucleotide variant being present in the cell-free sample of the subject, and wherein generating the likelihood value comprises learning a probability density function over a set of weights of the trained neural network.

28. The method of claim 27, wherein learning the probability density function comprises applying Bayesian inference to the set of weights.

29. The method of claim 28, wherein the trained neural network comprises a BiLSTM RNN comprising two layers of BiLSTM cells.

30. The method of claim 22, wherein (b) comprises processing a sequence alignment map (SAM) or binary alignment map (BAM) file of the plurality of sequencing reads by: splitting the SAM or BAM file into a plurality of distinct microSAM or microBAM files, wherein each distinct microSAM or microBAM file comprises a different set from among a plurality of sets of the sequencing reads, and performing parallel processing of the plurality of distinct microSAM or microBAM files.
